(19) United States
(12) Patent Application Publication
(10) Pub. No.: US 2024/0399053 A1
(43) Pub. Date: Dec. 5, 2024

(54) HIGH-DOSE DRUG DELIVERY DEVICE

(71) Applicant: Biograil APS, Roskilde (DK)

(72) Inventors: Nikolaj Skak, Roskilde (DK); Karsten Lindhardt, Roskilde (DK); Kamille Majken Dumong Erichsen, Roskilde (DK); Henrik Bang Mikkelsen, Roskilde (DK)

(21) Appl. No.: 18/698,322

(22) PCT Filed: Oct. 28, 2022
(86) PCT No.: PCT/EP2022/080195

§ 371 Date: Apr. 3, 2024

(30) Foreign Application Priority Data
Oct. 28, 2021 (DK) PA202170524

(51) Int. Cl. A61M 5/142; A61M 5/145; A61M 5/155; A61M 5/162; A61M 31/00

(52) U.S. Cl. CPC A61M 5/14276; A61M 5/14586; A61M 5/155; A61M 5/162; A61M 31/002; A61M 2005/14506; A61M 2205/04

(57) ABSTRACT

A high-dose drug delivery device comprising a first body part, a second body part, an internal chamber within the first body part, an actuator mechanism configured to rotate at least one of the first body part or the second body part with respect to one another about a central axis of the high-dose drug delivery device, a first attachment part in communication with the internal chamber, and an injector mechanism configured to apply pressure on the internal chamber during or after rotation of the first body part or the second body part with respect to one another.

The present disclosure relates to a drug delivery device and in particular to a drug delivery device for oral administration. The drug delivery device is advantageously configured for delivery of an active drug substance in the gastrointestinal tract including the stomach and/or intestines, such as the small intestines and/or the large intestines (colon).

### BACKGROUND

A number of for example low permeable and/or low water soluble active drug substances are currently delivered by i.e. subcutaneous, intradermal, intramuscular, rectal, vaginal or intravenous route. Oral administration has the potential for the widest patient acceptance and thus attempts to deliver low permeable and/or low water soluble active drug substances through the preferred oral route of administration has been tried but with limited success in particular due to lack of stability and limited absorption from the gastrointestinal tract.

Stability both relates to the stability of the active drug substance during manufacturing and storage of the delivery device, and to the stability of the active drug substance during the passage in the gastrointestinal tract before it become available for absorption.

Limited gastrointestinal absorption is due to the gastrointestinal wall barrier preventing active drug substance from being absorbed after oral dosing because of the low permeability of the active drug substance, which is for example due to pre-systemic metabolism, size and/or the charges and/or because of the water solubility of the active drug substance.

Multiple approaches to solve these stability and absorption challenges have been suggested, but an effective solution to the challenges remain unresolved.

### SUMMARY

Thus, there is an unmet need to provide a drug delivery device, which is capable of delivering drug substances for absorption in the gastrointestinal tissue. More generally, there remains a need for drug products and methods that enable enhanced drug delivery, when drug products are administered orally to patients.

Disclosed herein are examples of a high-dose drug delivery device. The high-dose drug delivery device comprises a first body part. The high-dose drug delivery device comprises a second body part. The high-dose drug delivery device comprises an internal chamber. The internal chamber can be within the first body part. The high-dose drug delivery device comprises an actuator mechanism. The actuator mechanism can be configured to rotate at least one of the first body part or the second body part with respect to one another about a central axis of the high-dose drug delivery device. The high-dose drug delivery device comprises a first attachment part. The first attachment part can be in communication with the internal chamber. The high-dose drug delivery device comprises an injector mechanism. The injector mechanism can be configured to apply pressure on the internal chamber during or after rotation of the first body part or the second body part with respect to one another.

It is an advantage of the present disclosure that the high-dose drug delivery device secures stability of the active drug substance during passage in the gastrointestinal tract and facilitates effective absorption of the active drug substance from the gastrointestinal tract after oral administration.

Further, it is advantage of the present disclosure that the high-dose drug delivery device provides an active attachment of the high-dose drug delivery device to the gastro-internal wall, such as the stomach wall and/or intestine wall.

Further, the present disclosure advantageously provides oral delivery of one or more of: low permeable, high permeable, low soluble, and high soluble active drug substances in or at the gastro-internal tissue. For example, the present disclosure can advantageously provide oral delivery of low soluble and high permeable active drug substances, low soluble and low permeable active drug substance, and/or high soluble and low permeable active drug substances. Further, the high-dose drug delivery device can advantageously deliver one or more of: liquid, semi-solid, and solid active drug substances. For example, the active drug substance can be solubilized, mixed, and/or dispersed in a liquid/semi-solid vehicle (for example water or a vehicle that melts at a temperature greater than room temperature but less than body temperature). A liquid formulation of an active drug substance may improve solubility/wettability.

Advantageously, the high-dose drug delivery device can provide fast delivery of an active drug substance. Further, advantageously the high-dose drug delivery device can store and/or provide large payloads of active drug substance. Advantageously, the high-dose drug delivery devices of the disclosure can effectively deliver large payloads of active drug substance. The high-dose drug delivery device can allow for improved distribution of an active drug substance, and faster, such as higher or more prolonged, absorption of the active drug substance.

Advantageously, the high-dose drug delivery device can allow for multiple types of dosing and/or larger payloads. For example, the high-dose drug delivery device can adjust pH at the delivery site. Further, the high-dose drug delivery device can use excipients to facilitate absorption and/or distribution of an active drug substance in tissue. Additionally, advantageously the high-dose drug delivery device can provide either, or both, of high dose (such as multi-mg) and low dose (such as sub-mg) active drug substances.

Moreover, the high-dose drug delivery device can allow for tonicity to control osmotic flow at a site of administration.

Further, the high-dose drug delivery device can advantageously allow for high tolerability in regard to tonicity, discomfort, and local tissue damage typically associated with injections, thus making administration easier.

### BRIEF DESCRIPTION OF THE DRAWINGS

The above and other features and advantages of the present invention will become readily apparent to those skilled in the art by the following detailed description of exemplary embodiments thereof with reference to the attached drawings, in which:

FIGS. 1A-1B illustrate a schematic of an example high-dose drug delivery device according to the disclosure,

FIGS. 2A-2B illustrate a schematic of an example high-dose drug delivery device according to the disclosure,

FIGS. 3A-3C illustrate a schematic of an example high-dose drug delivery device according to the disclosure,

FIGS. 4A-4C illustrate a schematic of an example high-dose drug delivery device according to the disclosure,

FIGS. 5A-5B illustrate a schematic of an example high-dose drug delivery device according to the disclosure,

FIG. 6 illustrates a schematic of a portion of an example high-dose drug delivery device according to the disclosure, and

FIGS. 7A-7B illustrate a schematic of a portion of an example high-dose drug delivery device according to the disclosure.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments and the functionalities associated therewith. They are not intended as an exhaustive description of the invention or as a limitation on the scope of the invention or the physical appearance of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

Disclosed herein are examples of a high-dose drug delivery device. The high-dose drug delivery device can comprise a first body part. The high-dose drug delivery device can comprise a second body part. The high-dose drug delivery device can comprise an internal chamber. The internal chamber can be within the first body part. The high-dose drug delivery device can comprise an actuator mechanism. The actuator mechanism can be configured to rotate at least one of the first body part or the second body part with respect to one another about a central axis of the high-dose drug delivery device. The high-dose drug delivery device can comprise a first attachment part. The first attachment part can be in communication with the internal chamber. The high-dose drug delivery device can comprise an injector mechanism. The injector mechanism can be configured to apply pressure on the internal chamber during or after rotation of the first body part or the second body part with respect to one another.

As discussed herein, the terms high-dose drug delivery device and drug delivery device can be used interchangeably.

The drug delivery device may have a size and geometry designed to fit into a pharmaceutical composition for oral administration.

The drug delivery device/pharmaceutical composition may be configured to be taken into the body via the oral orifice. Thus, the outer dimensions of the drug delivery device/pharmaceutical composition may be small enough for a user to swallow. The drug delivery device may be adapted to carry a drug substance into the body of the user, via the digestive system, so that the drug delivery device may e.g. travel from the mouth of the user into the stomach, via the esophagus. The drug delivery device may further travel into the intestines from the stomach, and may optionally travel into the bowels and out through the rectum.

The drug delivery device may be configured to deliver the drug in any part of the digestive system of the user, where in one example it may be configured to deliver a drug substance into the stomach of the user. In another example, the drug delivery device may be adapted to initiate the drug delivery when the device has passed the stomach and has entered the intestine of the user. In other words, the drug delivery device may be configured to attach to a wall of the stomach or a wall of the intestines, e.g. depending on the desired release position of the active drug substance.

The attachment part(s) of the drug delivery device may be configured to interact with the inner surface linings of the gastrointestinal tract, so that the drug delivery device may e.g. be attached to the inner surface (mucous membrane) of the stomach, or alternatively to the mucous membrane of the intestines. The attachment part(s) may be configured to interact with the mucous membranes, e.g. in order to fix or attach the drug delivery device, e.g. for a period of time, inside the body of the user. By attaching the drug delivery device, it will allow a drug substance to be delivered into a part of the digestive system, in order to provide a drug substance to the body of the user. The attachment part(s) may be configured to interact with the mucous membranes, e.g. in order to inject drug substance into the gastrointestinal tract wall.

The drug delivery device has a central axis optionally extending from a first end to a second end of the drug delivery device. The drug delivery device may have a length (e.g. largest extension from first end to second end along central axis), in the range from 3 mm to 35 mm, such as in the range from 5 mm to 26 mm. The drug delivery device may be elongated.

The drug delivery device may have a width and/or height (e.g. largest extensions along width axis and height axis, respectively) in the range from 1 mm to 20 mm. Height and width are the largest extensions of the drug delivery device perpendicular to the central axis.

In one or more exemplary drug delivery devices, the dimensions of the drug delivery device, at least in an initial state or first state prior to actuation of the first attachment part and/or the second attachment part, may be represented by a length (largest extension along central axis), a width (largest extension along width axis perpendicular to the central axis) and a height (largest extension along height axis perpendicular to the central axis and the width axis). The height of the drug delivery device may be in the range from 1 mm to 15 mm. The width of the drug delivery device may be in the range from 1 mm to 15 mm.

In one or more exemplary drug delivery devices, the drug delivery device may be constructed in a way that secures the drug delivery part to deliver a payload or active drug substance into the internal tissue or internal surface for distribution of the active drug substance in the subject through the blood vessels.

Advantageously, the drug delivery device may be attached, and may deliver the active drug substance, to a particular location in a patient's intestinal wall. Of course, the delivery device may be attached, and may deliver the active drug substance, to other places as well. In one or more exemplary drug delivery devices, the drug delivery device, such as the spike, may penetrate the muscularis mucosa. In one or more exemplary drug delivery devices, the drug delivery device, such as the spike, may not penetrate the muscularis externa. In one or more exemplary drug delivery devices, the spike may be positioned in the submucosa. In one or more exemplary drug delivery devices, the spike may be positioned in the submucosa parallel to the gut wall.

The drug delivery device comprises a first body part. The first body part may be a two-part body part, i.e. the first body part may comprise a first primary body part and a first secondary body part. The first body part has an outer surface. A first primary recess and/or a first secondary recess may be formed in the outer surface of the first body part.

The drug delivery device optionally comprises a shell having a first shell part. An outer surface of the first body part may constitute at least a part of the first shell part.

The drug delivery device comprises a first attachment part. The first attachment part may comprise a first base part and/or a first needle, e.g. a spike. The first attachment part has a first proximal end and a first distal end. The first attachment part, such as the first needle or spike, optionally has or extends along a first attachment axis. A first tip of the first needle forms the first distal end. In other words, the first distal end is a first tip of the first needle. The first base may be arranged at or constitute the first proximal end of the first attachment part. The first needle may have a length in the range from 1 mm to 15 mm such, as in the range from 3 mm to 10 mm. Thereby sufficient penetration into the internal tissue may be provided for while at the same time reducing the risk of damaging the internal tissue. The first distal end of the first attachment part may be provided with a tip configured to penetrate a biological tissue. The first distal end of the first attachment part may be provided with a gripping part configured to grip a biological tissue.

The first needle may have a cross-sectional diameter in the range from 0.1 mm to 5 mm, such as in the range from 0.5 mm to 2.0 mm.

The first needle may be straight and/or curved. The first needle may comprise a first primary section that is straight. The first needle may comprise a first secondary section, e.g. between the first primary section and the first distal end or between the first base and the first primary section. The first secondary section may be curved.

The first needle may include two or more straight portions formed at an angle. For example, the first needle may have a proximal portion that extends at a first angle from a connection point to the drug delivery device and a distal portion that extends at a second angle from a connection point to the drug delivery device. The first angle and the second angle may be different. The proximal portion may connect to the distal portion at a joint (e.g., bend, connection, angle) and have a joint angle between the proximal portion and the distal portion. The joint angle may be an acute angle, an obtuse angle, or a right angle. The angle may be, for example, 10, 20, 30, 40, 50, 60, 70, 80, 90, 100, 120, 130 140, 150, 160, or 170 degrees. This can advantageously allow for different angles of attach when the first needle interacts with inner surface linings. This may allow for improved attachment of the drug delivery device, while helping to reduce or avoid tissue damage. Further, the joint may be flexible. Alternatively, the joint may not be flexible.

The joint may be located at a center, or generally at a center, of a length of the first needle. Alternatively, the joint may be located 40, 45, 55, 60, or 65% up a length of the first needle from the proximal end.

In one or more exemplary first attachment parts, the first needle may have three, four, or five different portions at different angles, each connected by a joint. In some iterations, any or all of the different portions may be straight or curved. Each joint may be flexible or not flexible.

The attachment parts of the drug delivery device may be seen as any kind of attachment parts that may be capable of attaching the drug delivery device to a biological tissue, such as a stomach wall, a wall of the bowels and/or intestines of a human or animal body. The attachment parts may be adapted to extend in a direction away from the central axis of the drug delivery device, and/or a central axis of the first attachment part. This may mean that the attachment part(s), e.g. at least in an activated state or second state of the drug delivery device, may extend in a direction away from a peripheral surface (in radial direction) of the first body part and/or the second body part, so that the attachment part extends farther in a radial direction than the peripheral or outer surface of the body part.

The first attachment part may be fixed or rotationally attached to the first body part.

In one or more exemplary drug delivery devices, the drug delivery device comprises a second body part. The second body part may be a two-part body part, i.e. the second body part may comprise a second primary body part and a second secondary body part. The second attachment part is optionally attached to the second body part. The second attachment part may be fixed or rotationally attached to the second body part. The second body part has an outer surface. A second primary recess and/or a second secondary recess may be formed in the outer surface of the second body part.

In one or more exemplary drug delivery devices, the actuator mechanism is configured to store energy. For example, the actuator mechanism can be configured to store one or more of: potential energy, chemical potential energy, including effervescent energy, spring energy, elastic potential energy, electrical potential energy, gravitational potential energy, thermal energy, and compression energy, such as from compressed air and/or expanding polymers. The particular type of stored energy is not limiting.

The actuator mechanism can be configured to convert, such as release, expel, unload, change, the stored energy to kinetic energy. For example, the actuator mechanism can be configured to convert the stored energy to kinetic energy via movement of the first body part or the second body part. The actuator mechanism can be configured to convert the stored energy to kinetic energy via movement of the first body part and/or the second body part with respect to one another. The actuator mechanism can be configured to convert the stored energy to kinetic energy via rotational movement of the first body part and/or the second body part with respect to one another. The actuator mechanism can be configured to convert the stored energy to kinetic energy via translational movement of the first body part and/or the second body part with respect to one another. For example, the first body part may be translated away from the second body part, which can force the attachment part(s) into tissue. The actuator mechanism can be configured to convert the stored energy to kinetic energy via translational movement and rotational movement of the first body part and/or the second body part with respect to one another.

In one or more example drug delivery devices, the actuator mechanism is configured to rotate at least one of the first body part or the second body part with respect to one another about a central axis of the high-dose drug delivery device.

In one or more example drug delivery devices, translational movement, such as longitudinal movement, of the first body part and the second body part can be configured to convert the stored energy to kinetic energy.

In one or more example drug delivery devices, release of the second body part from the first body part can be configured to convert the stored energy to kinetic energy.

In one or more example drug delivery devices, the actuator mechanism can be configured to convert the stored energy to the kinetic energy by rotating at least one of the first body part or the second body part with respect to one another.

In one or more example drug delivery devices, the actuator mechanism can be configured to rotate the first body part and/or the second body part with respect to each other. In one or more exemplary drug delivery devices, the actuator mechanism is configured to rotate the first body part in relation to the second body part about a primary axis of the drug delivery device. The primary axis may be parallel to and/or coinciding with the central axis. Alternatively, or in conjunction, the actuator mechanism can be configured to non-rotationally translate the first body part in relation to the second body part.

In one or more exemplary drug delivery devices, the first body part is configured to rotate in a first direction and/or the second body part is configured to rotate in a second direction opposite to the first direction. For example, this may occur due to the conversion of the stored energy to kinetic energy.

The drug delivery device may comprise a frame part, where different parts, such as the first body part and/or the second body part are attached, e.g. fixed or rotatably attached to the frame part. In one or more exemplary drug delivery devices, the actuator mechanism, or parts thereof, may be attached to the frame part. Thereby, separate rotation of the first body part and the second body part in relation to the frame part may be provided for

The rotational connection between the first body part and the second body part allows the first body part to rotate relative to the second body part, without the two parts separating from each other before the attachment part(s) interact with the internal tissue, such as mucous membranes. Such a connection may be obtained in a plurality of ways, where in one example the first body part has a plug connection and the second body part has a socket connection, where this plug and socket configuration allows the first body part to rotate relative to the second body part. A second example could be to provide an axle that may be coaxial with the central axis and/or the primary axis, where the first body part and the second body part are configured to receive the axle, and a stopping device is arranged at first and second ends of the axle, on each side of the combined first and second body part, preventing the first body part and the second body part to slide in a longitudinal direction along the axle. The axle may be integrated in the first body part or in the second body part.

If an axle were used, the axle can be made of any number of different materials. For example, the axle can be made of metals and/or alloys and/or polymers and/or composites and/or composites and/or combinations thereof.

The first and/or the second body part may be arranged to rotate freely relative to each other, e.g. at least in the second state, and thereby allowing the attachment parts to rotate relative to each other. Thus, the attachment parts may be adapted to come into contact and/or penetrate tissue of the gastrointestinal tract. The rotation of the body parts relative to each other using a resilient force may move the attachment parts in such a way that they are capable of e.g. penetrating or pinching the mucous membrane in order to fix the drug delivery device at a location in the gastrointestinal tract, such as the stomach or intestines. The penetrating and/or pinching force may come from the actuator mechanism/resilient part, where the resilient part may be adapted to store a resilient force that is capable of forcing the attachment parts towards each other when the resilient force of the resilient part has been at least partly unleashed. The resilient part may e.g. be in the form of a spring or spring element, for example a torsional spring or a power spring, where the spring may be wound up to store mechanical energy, where the mechanical energy may be transmitted to the first and/or the second body part. When the mechanical energy is released, the first body part may rotate relative to the second body part, and where the mechanical energy may be transferred into the attachment parts via the body parts.

Within the context of the present description the term “rotational force” may be seen as Torque, moment, moment of force, rotational force or “turning effect”. Another definition of the term “rotational force” may be the product of the magnitude of the force and the perpendicular distance of the line of action of force from the axis of rotation. The rotational force may be seen as the force which is transferred from the resilient part to the attachment members of the drug delivery device via the body parts.

The conversion to kinetic energy, such as the rotational force may be defined as being large enough to penetrate into the gastrointestinal tissue. When the rotational force is applied to both the first and the second body part, the first attachment member may come into contact with the surface to be attached to, and where the rotational force applied to the second body part may cause the second attachment part to come into contact with the same surface, where the first attachment part provides a force, while the second attachment part provides a counter force to the first attachment part, so that the force is applied in such a manner that the first attachment part is forced in a direction towards the second attachment part, or vice versa. Translational force can be used as well.

In one or more exemplary drug delivery devices, a distance between the first attachment axis of the first attachment part and the primary axis, e.g. at least in an activated state or second state of the drug delivery device and optionally in an initial state of the drug delivery device, is larger than 0.5 mm.

In one or more exemplary drug delivery devices, a distance between the second attachment axis of the second attachment part and the primary axis, e.g. at least in an activated state or second state of the drug delivery device and optionally in an initial state of the drug delivery device, is larger than 0.5 mm.

In one or more exemplary drug delivery devices, the first attachment part is rotationally attached to the first body part, e.g. via a first joint connection having a first rotation axis. In other words, the first attachment part is optionally configured to rotate about a first rotation axis, e.g. in relation to the first body part. The first rotation axis may be parallel to the central axis and/or the primary axis. The first rotation axis may form a first angle with the central axis and/or the primary axis. The first angle may be less than 15°. The first angle may be in the range from 75° to 105°, such as 90°±5° or 90°.

In one or more exemplary drug delivery devices, the first body part may define a first body recess (e.g., cavity, slot, hole) extending to an outer surface of the first body part. The first body recess may be formed by solid walls on all sides except an outermost surface which is open. The first attachment part may be rotationally connected within the first body recess along a first attachment part axis. The first attachment part axis may be, for example, a pin (e.g., arm, support). The first attachment part axis may be parallel to the central axis and/or the primary axis. The first attachment part axis may be angled with respect to the central axis and/or the primary axis. Accordingly, the first attachment part may be configured to rotate within the recess along the first attachment part axis. Further, rotation of the first attachment part may be stopped at end surfaces of the recess.

The first body recess may extend along a portion of the outer surface of the first body. The first body recess may extend fully along an outer circumference of the first body. The first body recess may extend around 10, 15, 20, 25, 30, 35, 40, 45, 50, 55, 60, 65, 70, 75, 80, 85, 90, 95, or 100% of an outer circumference of the first body. The first body recess may extend around greater than 10, 15, 20, 25, 30, 35, 40, 45, 50, 55, 60, 65, 70, 75, 80, 85, 90, or 95% of an outer circumference of the first body. The first body recess may extend around less than 10, 15, 20, 25, 30, 35, 40, 45, 50, 55, 60, 65, 70, 75, 80, 85, 90, 95, or 100% of an outer circumference of the first body. The first body may optionally contain more than one first body recess, for example of a plurality of first attachment parts are used on the first body. If more than one first body recess is used, they may be spaced longitudinally apart and/or circumferentially apart.

The first body recess may extend from an outer surface toward the central axis through 5, 10, 15, 20, 25, 30, 35, or 40% of the drug delivery device. The first body recess may extend from an outer surface toward the central axis through greater than 5, 10, 15, 20, 25, 30, 35, or 40% of the drug delivery device. The first body recess may extend from an outer surface toward the central axis through less than 5, 10, 15, 20, 25, 30, 35, or 40% of the drug delivery device.

In one or more exemplary drug delivery devices, the first body recess may extend circumferentially, or partially circumferentially around the first body with the central axis being the longitudinal direction. The first body recess may extend perpendicularly with respect to the central axis and/or the primary access (e.g., may extend along a cross section of the drug delivery device perpendicular to the central axis and/or the primary access). The first body recess may have any number of shapes. For example, the first body recess can be a portion of a circle, such as a half circle. The first body recess can be a triangle. The first body recess can be a sector of a circle. The first body recess can be a curved edge connected by two straight edge. The first body recess can be two curved edges connected to each other by two straight edges.

Thus, the first attachment part may rotate on the first attachment part axis in order to move perpendicular to the central axis and/or the primary axis. In certain embodiments, the first attachment part may rotate at an angle between perpendicular and parallel with respect to the central axis and/or the primary axis.

In one or more exemplary drug delivery devices, when the first body part and/or the second body part rotate with respect to one another, the first attachment part and/or the second attachment part can rotate out of their respective recesses (e.g., first body recess and second body recess) due to the rotation of the first body part and/or the second body part. The continued rotation of the first body part and/or the second body part then causes the first attachment part and/or the second attachment part to pierce tissue to hold the drug delivery device in place.

In one or more exemplary drug delivery devices, the first attachment part extends, e.g. at least in an activated state of the drug delivery device and optionally in an initial state of the drug delivery device, in a direction away from the first body part. In other words, the first needle may extend, e.g. at least in an activated state of the drug delivery device and optionally in an initial state, from an outer surface of the first body part. Formulated differently, the first attachment axis may, e.g. at least in an activated state of the drug delivery device and optionally in an initial state of the drug delivery device form an angle of at least 45° with the central axis and/or the primary axis. An attachment part extending in a direction is to be understood as the direction from proximal end of attachment part/needle part to distal end of attachment part along the attachment axis of the attachment part.

The first attachment part may in a first state of the drug delivery device extend in a first primary direction and in a second state of the drug delivery device extend in a first secondary direction. The first primary direction and the first secondary direction may form an angle of at least 30°. The first primary direction may be parallel or substantially parallel to the central axis. The first primary direction may form an angle less than 60° with the central axis. The first secondary direction may form an angle of at least 60° such as about 90° with the central axis. The first secondary direction may be perpendicular to the central axis.

The first distal end of the first attachment part may be configured to move or be moved from a first primary position in a first state of the drug delivery device to a first secondary position in the second state.

The drug delivery device comprises a second attachment part. The second attachment part may comprise a second base part and/or a second needle, e.g. spike. The second attachment part has a second proximal end and a second distal end. The second attachment part, such as the second needle, optionally has or extends along a second attachment axis. A second tip of the second needle forms the second distal end. In other words, the second distal end is a second tip of the second needle. The second base may be arranged at or constitute the second proximal end of the second attachment part. The second needle may have a length in the range from 1 mm to 15 mm such, as in the range from 3 mm to 10 mm. Thereby sufficient penetration into the internal tissue may be provided for while at the same time reducing the risk of damaging the internal tissue. The second distal end of the second attachment part may be provided with a tip configured to penetrate a biological tissue. The second distal end of the second attachment part may be provided with a gripping part configured to grip a biological tissue.

The second needle may have a cross-sectional diameter in the range from 0.1 mm to 5 mm, such as in the range from 0.5 mm to 2.0 mm.

The second needle may be straight and/or curved. The second needle may comprise a second primary section that is straight. The second needle may comprise a second secondary section, e.g. between the second primary section and the second distal end or between the second base and the second primary section. The second secondary section may be curved.

The second needle may include two or more straight portions formed at an angle. For example, the second needle may have a proximal portion that extends at a first angle from a connection point to the drug delivery device and a distal portion that extends at a second angle from a connection point to the drug delivery device. The first angle and the second angle may be different. The proximal portion may connect to the distal portion at a joint (e.g., bend, connection, angle) and have a joint angle between the proximal portion and the distal portion. The joint angle may be an acute angle, an obtuse angle, or a right angle. The angle may be, for example, 10, 20, 30, 40, 50, 60, 70, 80, 90, 100, 120, 130 140, 150, 160, or 170 degrees. This can advantageously allow for different angles of attach when the second needle interacts with inner surface linings. This may allow for improved attachment of the drug delivery device, while helping to reduce or avoid tissue damage. Further, the joint may be flexible. Alternatively, the joint may not be flexible.

The joint may be located at a center, or generally at a center, of a length of the second needle. Alternatively, the joint may be located 40, 45, 55, 60, or 65% up a length of the second needle from the proximal end.

In one or more exemplary second attachment parts, the second needle may have three, four, or five different portions at different angles, each connected by a joint. In some iterations, any or all of the different portions may be straight or curved. Each joint may be flexible or not flexible.

In one or more exemplary drug delivery devices, both the first needle and the second needle include a joint. However, only one of the first needle and the second needle may include a joint with the other being straight and/or curved. If both the first needle and the second needle include a joint, the first distal tip and the second distal tip may be angled towards one another in order to facilitate attachment when the first body part and the second body part rotate with respect to one another.

In one or more exemplary drug delivery devices, the second attachment part is rotationally attached to the second body part, e.g. via a second joint connection having a second rotation axis. In other words, the second attachment part is optionally configured to rotate about a second rotation axis, e.g. in relation to the second body part. The second rotation axis may be parallel to the central axis and/or the primary axis. The second rotation axis may form a second angle with the central axis and/or the primary axis. The second angle may be less than 15°. The second angle may be in the range from 75° to 105°, such as 90°±5° or 90°.

In one or more exemplary drug delivery devices, the second body part may define a second body recess (e.g., cavity, slot, hole) extending to an outer surface of the second body part. The second body recess may be formed by solid walls on all sides except an outermost surface which is open. The second attachment part may be rotationally connected within the second body recess along a second attachment part axis. The second attachment part axis may be, for example, a pin (e.g., arm, support). The second attachment part axis may be parallel to the central axis and/or the primary axis. The second attachment part axis may be angled with respect to the central axis and/or the primary axis. Accordingly, the second attachment part can be configured to rotate within the recess along the second attachment part axis. Further, rotation of the second attachment part may be stopped at end surfaces of the second body recess.

The second body recess may extend along a portion of the outer surface of the second body. The second body recess may extend fully along an outer circumference of the second body. The second body recess may extend around 10, 15, 20, 25, 30, 35, 40, 45, 50, 55, 60, 65, 70, 75, 80, 85, 90, 95, or 100% of an outer circumference of the second body. The second body recess may extend around greater than 10, 15, 20, 25, 30, 35, 40, 45, 50, 55, 60, 65, 70, 75, 80, 85, 90, or 95% of an outer circumference of the second body. The second body recess may extend around less than 10, 15, 20, 25, 30, 35, 40, 45, 50, 55, 60, 65, 70, 75, 80, 85, 90, 95, or 100% of an outer circumference of the second body. The second body may optionally contain more than one second body recess, for example of a plurality of second attachment parts are used on the second body. If more than one second body recess is used, they may be spaced longitudinally apart and/or circumferentially apart.

The second body recess may extend from an outer surface toward the central axis through 5, 10, 15, 20, 25, 30, 35, or 40% of the drug delivery device. The second body recess may extend from an outer surface toward the central axis through greater than 5, 10, 15, 20, 25, 30, 35, or 40% of the drug delivery device. The second body recess may extend from an outer surface toward the central axis through less than 5, 10, 15, 20, 25, 30, 35, or 40% of the drug delivery device.

In one or more exemplary drug delivery devices, the second body recess may extend circumferentially, or partially circumferentially around the second body with the central axis being the longitudinal direction. The second body recess may extend perpendicularly with respect to the central axis and/or the primary access (e.g., may extend along a cross section of the drug delivery device perpendicular to the central axis and/or the primary access). The second body recess may have any number of shapes. For example, the second body recess can be a portion of a circle, such as a half circle. The second body recess can be a triangle. The second body recess can be a sector of a circle. The second body recess can be a curved edge connected by two straight edge. The second body recess can be two curved edges connected to each other by two straight edges.

Thus, the second attachment part may rotate on the second attachment part axis in order to move perpendicular to the central axis and/or the primary axis. In certain embodiments, the second attachment part may rotate at an angle between perpendicular and parallel with respect to the central axis and/or the primary axis.

In one or more exemplary drug delivery devices, when the first body part and/or the second body part rotate with respect to one another, the first attachment part and/or the second attachment part can rotate out of their respective recesses (e.g., first body recess and second body recess) due to the rotation of the first body part and/or the second body part. The continued rotation of the first body part and/or the second body part then causes the first attachment part and/or the second attachment part to pierce tissue to hold the drug delivery device in place.

In one or more exemplary drug delivery devices, the second attachment part extends, e.g. at least in an activated state of the drug delivery device and optionally in an initial state of the drug delivery device, in a direction optionally away from the second body part. In other words, the second needle may extend, e.g. at least in an activated state of the drug delivery device and optionally in an initial state, from an outer surface of the second body part. Formulated differently, the second attachment axis may, e.g. at least in an activated state of the drug delivery device and optionally in an initial state of the drug delivery device form an angle of at least 45° with the central axis and/or the primary axis.

The second attachment part may in a first state of the drug delivery device extend in a second primary direction and in a second state of the drug delivery device extend in a second secondary direction. The second primary direction and the second secondary direction may form an angle of at least 30°. The second primary direction may be parallel or substantially parallel to the central axis. The second primary direction may form an angle less than 60° with the central axis. The second secondary direction may form an angle of at least 60°, such as about 90° with the central axis. The second secondary direction may be perpendicular to the central axis.

The second distal end of the second attachment part may be configured to move or be moved from a second primary position in a first state of the drug delivery device to a second secondary position in the second state.

The drug delivery device comprises an actuator mechanism. The actuator mechanism can be configured to store energy. The actuator mechanism can be configured to convert the stored energy to kinetic energy. For example, the actuator mechanism is configured to move the first attachment part in relation to the second attachment part, such as configured to move the first distal end towards and/or away from the second distal end, e.g. at least during a part of a rotation and/or a translation, such as in a first rotation and optionally in a second rotation. To move the first distal end towards the second distal end may be understood as reducing a distance between the first distal end and the second distal end. To move the first distal end towards the second distal end may be understood as reducing an angle between the first attachment axis and the second attachment axis, such as reducing an angle between a first secondary direction of the first attachment part and a second secondary direction of the second attachment part. In one or more exemplary drug delivery devices, the actuator mechanism is configured convert stored energy to kinetic energy to move the first distal end towards the second distal end by rotating, e.g. in a second state of the drug delivery device, the first body part in relation to the second body part and/or vice versa. The actuator mechanism may be configured to convert stored energy to kinetic energy to rotate the first body part at least 90°, such as at least 450°, at least 810°, at least 1170°, at least 1530°, or even at least 1890° in relation to the second body part about the primary axis. The actuator mechanism may be configured to convert stored energy to kinetic energy to rotate the first body part in relation to the second body part about the primary axis in a stepwise manner. In other words, to rotate the first body part in relation to the second body part about the primary axis may comprise a plurality of rotations including a first rotation and a second rotation, e.g. a first rotation followed by a first time period with reduced or no rotation followed by a second rotation. A first rotation followed by a second rotation after a first time period may increase the possibility of the drug delivery device attaching to the biological tissue. The first time period or in general time periods between rotations allows the drug delivery to move to other positions in the gastrointestinal tract. In other words, if the drug delivery does not attach to the biological tissue during a first rotation, further rotations increase the chance of attachment to the internal tissue. The first rotation may be at least 90°, and the second rotation may be at least 180°. The plurality of rotations may comprise a third rotation. The third rotation may be at least 180°.

In one or more exemplary drug delivery devices, a movement of the first distal end towards the second distal end may be preceded by and/or followed by a movement of the first distal end away from the second distal end. In other words, a movement of the first distal end towards the second distal end may be prior to and/or after movement of the first distal end away from the second distal end. For example, a first rotation may comprise moving the first distal end towards the second distal end and/or moving the first distal end away from the second distal end. For example, a second rotation may comprise moving the first distal end towards the second distal end and/or moving the first distal end away from the second distal end. For example, a third rotation may comprise moving the first distal end towards the second distal end and/or moving the first distal end away from the second distal end.

The actuator mechanism optionally comprises a resilient part such as a spring element configured to apply force to the first body part and/or the second body part. The spring element may be configured to store energy, such as elastic potential energy. The resilient part may comprise a first part, such as a first end, connected to the first body part. The resilient part may comprise a second part, such as a second end, connected to the second body part.

In one or more exemplary drug delivery devices, the actuator mechanism optionally comprises a swelling media, i.e. a media increasing its volume, e.g. upon contact with a fluid, e.g. in order to provide rotation of parts in relation to each other. In one or more exemplary drug delivery devices, a swelling medial provides rotation of the first attachment part in relation to the first body part and/or provides rotation of the second attachment part in relation to the second body part. In one or more exemplary drug delivery devices, a swelling media provides rotation of the first body part in relation to the second body part. The swelling media can similarly be configured to store energy and convert the stored energy to kinetic energy.

The actuator mechanism, such as the resilient part, may be configured to rotate the first attachment part about the first rotation axis in relation to the first body part, for example during the conversion of the stored energy to the kinetic energy.

The actuator mechanism, such as the resilient part, may be configured to rotate the second attachment part about the second rotation axis in relation to the second body part, for example during the conversion of the stored energy to the kinetic energy.

In one or more exemplary drug delivery devices, the drug delivery device can include an internal chamber, such as a compartment, container, space, volume. In one or more exemplary drug delivery devices, the drug delivery device comprises an internal chamber, the drug delivery device being configured to deliver an active drug substance from the internal chamber to the surroundings of the drug delivery device.

The internal chamber may be within the first body part. The internal chamber may be arranged within the first body part. The internal chamber may be located within the first body part. The internal chamber may be within the second body part. The internal chamber may be arranged within the second body part. The internal chamber may be located within the second body part. The internal chamber may be located within both the first body part and the second body part.

The internal chamber may be multiple chambers. For example, the internal chamber may be a first internal chamber and a second internal chamber. The internal chamber may include other chambers within, such as a primary chamber and/or secondary chamber.

The internal chamber may be portioned into different chambers, such as different chamber sections and/or different chamber portions.

The internal chamber may contain, or be configured to contain, a container. A contain can be, for example, a pouch, a bag, a balloon, etc.

The first attachment part, such as the first needle may have one or more openings providing access to the internal chamber. In one or more exemplary drug delivery devices, the internal chamber is connected external the drug delivery device via a through-going bore in the first needle. The first attachment part can be in communication with the internal chamber. The first attachment part can be in fluid communication with the internal chamber. The first attachment part can provide fluid connecting between the internal chamber and an outside of the drug delivery device.

As the active drug substance may not always be a fluid, such as a semi-fluid and/or a solid, the first attachment part can be in communication with the internal chamber to allow the active drug substance, regardless of the state, to pass through the first attachment part and exit the drug delivery device. Fluid communication, as discussed herein, can also allow a solid or semi-solid active drug substance, or other substance in the drug delivery device, to proceed from the internal chamber, through the first attachment part, and outside of the drug delivery device.

In one or more example drug delivery systems, the first attachment part can include a channel. The channel can be fluidly connecting the internal chamber and an outer surface of the first attachment part. The first attachment part can include a channel, or lumen, tunnel, connecting, such as fluidly connecting, the internal chamber and an outside of the drug delivery device. The channel may be blocked, such as closed, stopped. This can prevent movement between the internal chamber and an outside of the drug delivery device. For example, the channel may be blocked by a biodegradable plug, such as a stopper. The attachment part may be blocked by one or more components of the drug delivery device before rotation by the actuator mechanism. For example, rotation of the first body part with respect to the second body part can open a fluid communication between the internal chamber and the first attachment part. The channel can extend along a length of the first attachment part. The first attachment part may have an aperture, such as leading to a channel in the first attachment part.

The first attachment part may have a portion, such as an end, located within the first body part and/or the second body part, such as within the drug delivery device. The portion of the first attachment part may include an edge, such as a piercing edge or a cutting edge. The edge may be an end of the first attachment part. The edge may not be an end of the first attachment part.

In one or more example drug delivery devices, the internal chamber can be configured to releasably retain an active drug substance. The drug delivery device may use a release mechanism to release the active drug substance.

In one or more example drug delivery devices, the active drug substance can be a solid. In one or more example drug delivery devices, the active drug substance can be a solid rod. In one or more example drug delivery devices, the active drug substance can be a liquid. In one or more example drug delivery devices, the active drug substance can be a suspension. In one or more example drug delivery devices, the active drug substance can be a liquid and/or suspension. In one or more example drug delivery devices, the active drug substance can be a gel. The particular type and/or state of the active drug substance is not limiting.

The internal chamber can further retain an excipient with the active drug substance. For example, the internal chamber can hold an active substance that can serve as a vehicle, such as a medium, for an active drug substance. This can allow for easier delivery of the active drug substance.

The internal chamber can further retain other substances than the active drug substance. For example, the internal chamber can include a substance to change pH.

For example, the internal chamber may include a first internal chamber holding an active drug substance and a second internal chamber holding a pH changing substance, such as a pH buffer or solution. This may allow for control of the pH of the microenvironment around any injection site.

The internal chamber may hold substances other than the active drug substance. For example, the substances can affect the microenvironment around any injection site. Different substances can be held in different chambers of the internal chamber.

The excipient and/or the pH changing substance may be included in a coating on the drug-delivery device.

In one or more example drug delivery devices, the internal chamber be configured to releasably retain 1 μg-500 mg of the active drug substance. For example, the internal chamber can be configured to releasably retain, or hold, at least 1, 2, 3, 4, 5, 6, 7, 8, 9, 10, 20, 30, 40, 50, 60, 70, 80, 90, 100, 200, 300, 400, or 500 μg. The internal chamber can be configured to releasably retain, or hold, at least 1, 2, 3, 4, 5, 6, 7, 8, 9, 10, 20, 30, 40, 50, 60, 70, 80, 90, 100, 200, 300, 400, or 500 mg.

The internal chamber may be arranged in any part of the drug delivery device, such as in the form of a cavity inside a volume of the first body part, the second body part or both of the first body part and the second body part.

In one or more exemplary drug delivery devices, the internal chamber may be open from an inner volume of the drug delivery device and towards an outer part of the drug delivery device. In one or more examples, the internal chamber may be inside the first body part, and where the internal chamber is in fluid connection with the first attachment part, so that when the first distal end of the first attachment part has penetrated the biological tissue, the drug substance may be released from the internal chamber and into the biological tissue via the first attachment part. This may be where the first attachment part is a tubular part, such as having a channel, which has a first distal end in fluid communication with the internal chamber of the drug delivery device.

The internal chamber can have a particular volume. For example, the internal chamber can have a volume of 1, 2, 3, 4, 5, 6, 7, 8, 9, 10, 20, 30, 40, 50, 60, 70, 80, 90, or 100 μL.

The internal chamber can have a volume of at least 1, 2, 3, 4, 5, 6, 7, 8, 9, 10, 20, 30, 40, 50, 60, 70, 80, 90, or 100 μL. The internal chamber can have a volume of less than 1, 2, 3, 4, 5, 6, 7, 8, 9, 10, 20, 30, 40, 50, 60, 70, 80, 90, or 100 μL. In one or more example drug delivery devices, the internal chamber can have a volume of at least 5 μL.

The internal chamber can have a volume of 0.5, 1, 2, 3, 4, 5, 6, 7, 8, 9, or 10 mL. The internal chamber can have a volume of less than 0.5, 1, 2, 3, 4, 5, 6, 7, 8, 9, or 10 mL. The internal chamber can have a volume of greater than 0.5, 1, 2, 3, 4, 5, 6, 7, 8, 9, or 10 mL. In one or more example drug delivery devices, the internal chamber can have a volume of at least 1 mL.

In one or more exemplary drug delivery devices, the drug delivery device can include an injector mechanism, such as an injector, and injector system, injector device, injector setup, injection mechanism. The injector mechanism can be configured to apply a force on and/or within the internal chamber. For example, the injector mechanism can be used to inject, such as expel, eject, force out, a substance out of the internal chamber. For example, the injector mechanism can inject a substance out of the internal chamber, through the first attachment part, and out of the drug delivery device. The injector mechanism can be configured to apply and/or create pressure on and/or in the internal chamber during rotation of the first body part or the second body part with respect to one another, or the conversion of stored energy to kinetic energy. The injector mechanism can be configured to apply pressure on the internal chamber after rotation of the first body part or the second body part with respect to one another, or the conversion of stored energy to kinetic energy. The injector mechanism can be configured to apply pressure on the internal chamber before rotation of the first body part or the second body part with respect to one another, or the conversion of stored energy to kinetic energy. The injector mechanism can be configured to apply a force, such as a compression force, on the internal chamber during or after rotation of the first body part or the second body part with respect to one another, or the conversion of stored energy to kinetic energy. The injector mechanism can be configured to move one or more components of the drug delivery device.

The drug delivery device may include a release mechanism. The drug delivery device may not include a release mechanism. The injector mechanism may be separate from a release mechanism. The injector mechanism may be a release mechanism. The injector mechanism may not be a release mechanism.

The injector mechanism can be one of many different types of injector mechanisms, including but not limited to those discussed herein. Further, the injector mechanism can include combinations of one or more of the following injector mechanisms.

The injector mechanism can be configured to quickly inject an active drug substance. For example, the injector mechanism can be configured to inject an active drug substance in less than 1, 2, 3, 4, 5, 6, 7, 8, 9, or 10 seconds. The injector mechanism can be configured to slowly inject the active drug substance. For example, the injector mechanism can be configured to inject the active drug substance over 1 hour, 1 day, 2 days, 3 days, or more. The active drug substance may be released upon a release mechanism being triggered.

In one or more example drug delivery devices, the injector mechanism may be a swellable polymer. For example, the internal chamber can include a swellable polymer and an active drug substance. The swellable polymer can be configured to swell, such as expand, grow, enlarge. The swellable polymer can be configured to swell upon contact of a particular substance, such as a liquid. For example, the substance, such as liquid, which can cause the swellable polymer to swell may be liquid from a stomach or intestines of a user. The liquid may come from a separate chamber in the drug delivery device.

The swellable polymer can be configured to immediately swell to a maximum size. The swellable polymer can be configured to swell at a particular rate. Example swellable polymers include, but are not limited to, one or more of: gellan gum, hydroxypropyl methylcellulose, polyethylene glycol, polyethylene oxide, polyoxyethylene, sodium polyacrylate, sodium starch glycolate, alginates, xanthan gum, locust bean gum, starches, polyvinylpyrrolidone, and ammonium methacrylate copolymer (Eudragit RS), ethyl cellulose, polyvinyl acrylate, polyvinylpyrrolidone, poly(vinyl alcohol), tragacanth gums, celluloses (e.g. carboxymethyl cellulose, hydroxypropyl cellulose), and microcrystalline cellulose.

The swelling of the swellable polymer in the internal chamber can increase the pressure within the internal cavity. When the active drug substance has a path outside of the internal chamber, such as via an opening of a release mechanism, the pressure caused by the swelling of the swellable polymer may allow the active drug substance to exit the internal chamber, such as to outside the drug delivery device. For example, if the active drug substance is a liquid, the swelling of the swellable polymer my cause the liquid to be under pressure. This may also apply to a semi-solid and/or a solid active drug substance.

Alternatively, the swellable polymer may swell to a degree that can physically interact with the active drug substance to translate it out of the internal chamber.

The active drug substance may be located within a container, such as a pouch or compartment. The swelling of the swellable polymer may push the container into a piercing component, such as a spike, needle, etc. which can release the active drug substance. The piercing component and container can be considered a release mechanism as well.

The swellable polymer may be within the internal chamber. The swellable polymer may be located within, for example, an expandable container which can expand under the forces exerted by the swellable polymer.

The swellable polymer may start swelling upon rotation of the first body part with respect to the second body part, or the conversion of stored energy to kinetic energy. For example, rotation may allow the swellable polymer to translate into the internal compartment so that it can expand. Rotation may allow a liquid to interact with the swellable polymer so that It begins to swell. For example, rotation may open a pathway for liquid to enter the drug delivery device and interact with the swellable polymer.

In one or more example drug delivery devices, the injector mechanism can include an expandable gas. The expandable gas can be a single gas. The expandable gas can be a mixture of gases. The expandable gas can be configured to expand immediately. The expandable gas can be configured to expand at a particular rate. The expanding of the expandable gas in the internal chamber can increase the pressure within the internal cavity. When the active drug substance has a path outside of the internal chamber, the pressure caused by the expanding of the expandable gas may allow the active drug substance to exit the internal chamber, such as to outside the drug delivery device. Alternatively, the expandable gas may expand to a degree that can physically interact with the active drug substance to translate it out of the internal chamber. Example expandable gases include, but are not limited to, one or more of carbon dioxide, air, helium, and oxygen.

For example, the expandable gas can be sealed within a secondary chamber within the internal chamber. The secondary chamber can be configured to degrade to release the expandable gas at a particular time, such as after a particular time. The secondary chamber may retain the expandable gas under pressure. The secondary chamber may not retain the expandable gas under pressure.

The active drug substance may be located within a container, such as a pouch or compartment. The expanding of the expandable gas may push the container into a piercing component, such as a spike, needle, etc. which can release the active drug substance. This can be an example of a release mechanism.

The expandable gas may be within the internal chamber. The expandable gas may be located within, for example, an expandable container which can expand under the forces exerted by the swellable polymer. This can increase the pressure in the internal chamber. This expansion of the expandable container can also push a container holding the active drug substance into a piercing component. This can be an example of a release mechanism.

The expandable gas may start expanding upon rotation of the first body part with respect to the second body part, or the conversion of stored energy to kinetic energy. For example, rotation may allow the expandable gas to translate into the internal compartment so that it can expand. The rotation may pierce a container holding the expandable gas. For example, the first attachment part having an edge may pierce the container.

In one or more example drug delivery devices, the injector mechanism can include a first component separated from a second component. The rotation, or the conversion of stored energy to kinetic energy, can interact the first component with the second component to form an expandable gas.

For example, the internal chamber, or first internal chamber, may retain a first component, such as a first gas or first substance, and the secondary chamber, or second internal chamber, may retain a second component, such as a second gas or a second substance. The first and second components may be separated. Upon release from the secondary chamber, or second internal chamber, the second component and the first component may interact causing the formation of an expandable gas which can expand. For example, rotation, or the conversion of stored energy to kinetic energy, can form a fluid connection channel. Rotation can break and/or pierce the first internal chamber and/or the secondary chamber, or secondary internal chamber. Rotation can open up a pathway between the first internal chamber and the secondary internal chamber.

As an example, the internal chamber may include a primary chamber separated from a secondary chamber. The primary chamber may hold the active drug substance. The secondary chamber may hold an injection mechanism. For example, the secondary chamber may hold a pressurized air pocket. Once released from the secondary chamber, the pressurized air pocket may still pressurize the internal chamber.

The secondary chamber may separately hold the first component and the second component. For example, the secondary chamber may be partitioned into a first secondary chamber and a second secondary chamber. The first secondary chamber may hold a first component, such as citric acid and calcium carbonate combination. The second secondary chamber may hold a second component, such as water. The first component can be released form the first secondary chamber into the second secondary chamber, or vice versa, which can cause a reaction to form an expandable gas. The expandable gas may then release from the secondary chamber.

In one or more example drug delivery devices, the first component can be separated from the second component via a plug, or stopper. The plug can be a pH releasable plug. The plug can be a pH dissolvable plug. In one or more example drug delivery devices, the first component is separated from the second component via a pH releasable plug. The pH releasable plug may dissolve after a certain time period.

The releasable plug may block the second component from releasing from the secondary chamber. The releasable plug may form a secondary chamber.

The first component can be a liquid and the second component can be the swellable polymer discussed above. The liquid can be separated from the swellable polymer until rotation, or the conversion of stored energy to kinetic energy, which can then allow the swellable polymer to swell.

In one or more example drug delivery devices, the injector mechanism can include a breakable seal. The breakable seal can be incorporated into the above-discussed injector mechanisms as well. The breakable seal may be used to contain an injector mechanism component. The breakable seal may, for example, contain the expandable gas. The breakable seal may be a breakable seal to the secondary contain. The breakable seal may contain the swellable polymer. The breakable seal may contain a substance configured to swell the swellable polymer. The breakable seal may hold the active drug substance in the internal chamber under pressure, so that when the breakable seal breaks the active drug substance can be released.

The breakable seal may seal the internal chamber. For example, the breakable seal may act as a release mechanism. The breakable seal may seal a secondary chamber within the internal chamber. The breakable seal may partition, such as segregate, one or more portions of the internal chamber.

The breakable seal may be broken in a number of ways. For example, the rotation of the at least one first body part or the second body part, or the conversion of stored energy to kinetic energy, may break the seal. For example, the rotational motion may break the breakable seal. The internal chamber may include a spike, needle, and/or puncturing component. Upon rotation, the spike, needle, and/or puncturing component may break the breakable seal. The breakable seal may be dissolvable, such as degradable or biodegradable.

The rotation may move the breakable seal from one position to another position to open up a fluid communication path between the internal chamber and an outside of the drug delivery device, thereby “breaking” the breakable seal.

In one or more example drug delivery devices, the injector mechanism can include a spike. The spike can be configured to pierce a container. The spike and/or container may be considered a release mechanism. The spike may be the first attachment part, such as the edge. The spike may not be the first attachment part. The container can be, for example, a pouch or bag. The container may contain an active drug substance. The active drug substance may be under pressure. The container may contain a substance to active a portion of the injector mechanism. The container can be a secondary chamber. The spike can contain an aperture and be configured to penetrate the container. The opening can be, for example, half-way along the spike. The spike may extend, and/or be a portion of, the first attachment part.

The container may be pierced in a number of ways. For example, the rotation of the at least one first body part or the second body part, or the conversion of stored energy to kinetic energy, may pierce the container. For example, the rotational motion may pierce the container. The internal chamber may include a spike, needle, and/or puncturing component. Upon rotation, the spike, needle, and/or puncturing component may pierce the container. The spike may be rotated to pierce the container. The container may be rotated into the spike. The injector mechanism may translate the container into the spike. The injector mechanism may translate the spike into the container.

In one or more example drug delivery devices, the injector mechanism can include a compression spring. The injector mechanism can include a spring. The injector mechanism can include a tension spring.

The injector mechanism can include a compression spring attached to a platform. Prior to rotation, or the conversion of stored energy to kinetic energy, the compression spring can be in a compressed position. After rotation, or the conversion of stored energy to kinetic energy, the compression spring can be allowed to uncompress, thereby translating the platform. The platform can be configured to define the internal chamber. When the platform is translated via the compression spring uncompressing, the platform can cause the internal chamber, such as including an active drug substance, to be under pressure. When the internal chamber is breached, the active drug substance can then be quickly injected. The compression spring may be in a compressed position to start. The compression spring may compress to apply pressure during use of the drug delivery device. The compression spring may compress to apply pressure when the drug delivery device converts the stored energy to kinetic energy, such as via rotation.

A container may be attached to the compression spring. The container may contain the active drug substance. The active drug substance may be under pressure. Upon uncompressing, the compression spring may cause the container to break, thereby releasing the active drug substance.

In one or more example drug delivery devices, the injector mechanism can be a thread mechanism. For example, it can be configured to move components longitudinally towards each other. For example, the thread mechanism can include a platform configured to define the internal chamber. As the platform translates along the thread mechanism, the platform can cause the internal chamber, such as including an active drug substance, to be under pressure. When the internal chamber is breached, the active drug substance can then be quickly injected.

The thread mechanism can cause the first body part and the first body part to translate closer towards one another. This movement can reduce the volume of the internal chamber, which can increase pressure in the internal chamber.

The injector mechanism may be configured to generate osmotic pressure. For example, a semi-permeable membrane can be included, which may slowly increase pressure in the internal chamber.

The injector mechanism may be initiated by a patient's body. For example, during rotation of the first body part and/or the second body part, or conversion of stored energy to kinetic energy, a cover, such as a seal, a shell, a capsule, can be released from the drug delivery device. The cover can cover a portion of the drug delivery device. When the cover is removed, the internal chamber may be exposed for compression by gastric forces and/or peristaltic forces. For example, the internal chamber may include an aperture which is covered by the cover.

In one or more example drug delivery devices, the internal chamber can be under pressure prior to the rotation. The internal chamber can be under pressure. The internal chamber can be pressurized. The internal chamber can be under pressure during the rotation. The internal chamber can be under pressure after the rotation. A container in the internal chamber can be under pressure prior to the rotation. For example, a secondary chamber and/or a second internal chamber can be under pressure prior to the rotation. A portion of the internal chamber may be under pressure prior to the rotation and a different portion of the internal chamber may not be under pressure prior to the rotation.

In one or more example drug delivery devices, the injector mechanism can be a compression source. In one or more example drug delivery devices, the injector mechanism can be a kinetic energy source. For example, the injector mechanism can be one or more of a spring, an osmotic mechanism, a swelling polymer, citric acid/calcium carbonate, stent, memory alloy, long axle, peristaltic compression, elastic bag, and pressurized air pocket.

In one or more example drug delivery devices, the internal chamber can be connected to the first attachment part. The active drug substance can be injected from the internal chamber via a single outlet, such as a channel. The channel can allow for liquid, semi-liquid, and/or solid active drug substances to pass through. The internal chamber can be connected to the first attachment part and a second attachment part. The active drug substance can be injected from the internal chamber via more than one outlet, such as more than one channel.

The drug delivery device may include a single internal chamber. The drug delivery device may include a plurality of internal chambers, such as a first internal chamber and a second internal chamber. The first attachment part may be connected to both the first internal chamber and the second internal chamber, such as via a channel and/or a plurality of channels. For example, the first internal chamber may retain a first active drug substance and the second internal chamber may retain a second active drug substance. The first active drug substance may be non-compatible with the second active drug substance. The first internal chamber may be breached first to release the first active drug substance. Subsequently, the second internal chamber may be breached to release the second active drug substance. Alternatively, a first attachment part may be connected with the first internal chamber and a second attachment part may be connected to the second internal chamber.

In one or more example drug delivery devices, the internal chamber can include a pouch. The pouch can be configured to contain the active drug substance. The pouch can be, for example, one or more of a conventional pouch, a harmonica pouch, a doughnut pouch, and a twisting pouch. The drug delivery device may not contain a pouch. The pouch may be under pressure. The injector mechanism can be configured to break, breach, and/or pierce the pouch. The release mechanism can be configured to break, breach, and/or pierce the pouch.

The internal chamber can be configured to retain one or more inserts. The inserts can be inserted into the internal chamber. The inserts can form a portion of the internal chamber. For example, the internal chamber can receive a first insert configured to hold an active drug substance. The second insert can be configured to hold the injector mechanism. The injector mechanism can be one or more of the above-described injector mechanisms, or any other injector mechanism. The second insert and the first insert can be configured to form a fluid communication between the two, such as after rotation or breakage of a seal separating the first insert and the second insert. The inserts can be breakable. The inserts can be pierceable. The inserts may form a first internal chamber and a second internal chamber. The first insert may be connected to the first attachment part and the second insert may be connected to the second attachment part.

In one or more example drug delivery devices, the injector mechanism can be a bag, such as a pouch. The bag can be located in the internal chamber. The rotation of the first body part and/or the second body part, or the conversion of stored energy to kinetic energy, provide a sufficient force to empty the bag. The bag can be detachable from the drug delivery device. For example, the bag may be released from the drug delivery device.

The injector mechanism can use osmotic pressure to increase the pressure within the internal chamber. For example, the injector mechanism can include an osmotic pump.

The injector mechanism can be a pressurizing mechanism. For example, the injector mechanism can be configured to pressurize the internal chamber.

The drug delivery device can include one or more release mechanisms for releasing the active drug substance from the internal chamber. For example, a release mechanism can allow for liquid to flow from the internal chamber and through the attachment part. The release mechanism may allow for the active drug substance to translate from the internal chamber to the attachment part and out of the drug delivery device. As discussed above, the release mechanism may be part of the injector mechanism. The release mechanism may be separate from the injector mechanism.

Different release mechanisms can be used, and the particular release mechanism is not limiting.

For example, a valve can be used as a release mechanism, which can open access, such as fluid communication, between the attachment part and the internal chamber. The valve can be a blocking valve. The valve can be a ball plug valve. The valve can be a ballo-fix valve. For example, when enough pressure is put on the internal chamber, this may activate the valve to open, allowing liquid to flow. As another example, rotation of the first body part and the second body part may open the valve.

As discussed, penetration of a container, such as a pouch, can be the release mechanism. For example, the container can be under pressure as discussed herein. The penetration can be by rotation of the first body part with respect to the second body part. Penetration can be by translation of the first body part with respect to the second body part. The penetration can be caused by the first attachment part. For example, as discussed, the first attachment part may have an edge.

Permeation of a container can be a release mechanism. Liquid flow into a cavity connecting the internal chamber with the attachment part can be a release mechanism. Release, such as removal, of a plug from an aperture can be a release mechanism. A permanent connection with a plug can be the release mechanism. For example, the plug may be dissolvable, such as a water soluble plug. The plug may be in the internal chamber. The plug may be in the attachment part. The plug may be in a connection between the attachment part and the internal chamber. The plug may be swellable.

In one or more example drug delivery devices, the release mechanism may not use the first attachment part. For example, the internal chamber can include an expandable container, such as an expandable pouch or balloon. As the expandable container expands, a release spike can be exposed from the internal chamber to external the device. The release spike may be attached to the expandable container. The release spike may provide a fluid communication between the internal chamber and an outside of the drug delivery device. For example, the release spike may penetrate an outer surface of the drug delivery device. The release spike may penetrate a sealed aperture.

The active drug substance can be located in the internal chamber. The active drug substance can be located in the first attachment part. The active drug substance can be located in both the internal chamber and the first attachment part. The injector mechanism can be configured to eject the active drug substance.

In one or more exemplary drug delivery devices, the drug delivery device comprises a second internal chamber, the drug delivery device being configured to deliver an active drug substance from the second internal chamber to the surroundings of the drug delivery device. The active drug substance in the second internal chamber may be the same as the active drug substance in the internal chamber. The active drug substance in the second internal chamber may be the different from the active drug substance in the internal chamber. The second internal chamber may be arranged in the first attachment part or in the second attachment part, such as in the second needle, e.g. within a distance of 8 mm, such as within 5 mm, from the second distal end. The second attachment part, such as the second needle may have one or more openings providing access to the second internal chamber. In one or more exemplary drug delivery devices, the second internal chamber is formed as a through-going bore in the first needle or in the second needle.

In one or more exemplary drug delivery devices, the first attachment part and the second attachment part form an angle when the first distal end and the second distal end are in a plane that includes the primary axis. In other words, the first attachment axis and the second attachment may form an angle, e.g. larger than 5°, such as in the range from 10° to 75°, when the first distal end and the second distal end are in a plane that includes the primary axis.

In one or more exemplary drug delivery devices, the drug delivery device has a first state, also denoted initial state, where the first body part and the second body part are rotationally stationary relative to each other and a second state, also denoted activated state, where the first body part and the second body part are rotationally mobile relative to each other, e.g. can rotate about the primary axis of the drug delivery device. In other words, the first body part may be locked, e.g. prevented from rotating, in relation to the second body part. The first state may e.g. be an initial state or introduction state, where the drug delivery device is adapted to be introduced into the body, and where the first body part and the second body part are stationary relative to each other. In the first state the resilient part may have a predefined amount of stored energy, where the energy level is stationary in the resilient part while the body parts are stationary. For example, the first state may occur when the actuator mechanism stores energy. The second state may occur when the actuator mechanism is converting the stored energy to kinetic energy.

In one or more exemplary drug delivery devices, the drug delivery device has a first state where the resilient part has a constant resilient force load, such as a stored energy, and a second state where the resilient part at least partly releases the resilient force load, such as at least partially converting the stored energy to kinetic energy. In other words, the resilient part may be biased or preloaded in the first state of the drug delivery, such as storing energy, and upon release, e.g. by release of a locking mechanism, (i.e. the drug delivery device being in the second state) the force from the resilient part may effect a rotation of the first body part in relation to the second body part, i.e. including a movement of the first distal end towards the second distal, such as a conversion of the stored energy to kinetic energy.

In one or more exemplary drug delivery devices, the actuator mechanism is configured to move the first distal end from a first primary position, e.g. in first state of the drug delivery device, with a first primary radial distance from a central axis of the delivery device to a first secondary position, e.g. in second state of the drug delivery device, with a first secondary radial distance from the central axis and/or primary axis, wherein the first secondary radial distance is larger than the first primary radial distance. Thus, the first distal end of the first attachment may be in a first primary position when the drug delivery device is in the first state and/or the first distal end of the first attachment part may be in a first secondary position when the drug delivery device is in the second state.

The first primary radial distance may be less than 10 mm, such as less than 8 mm or even less than 5 mm. The first secondary radial distance may be larger than the first primary radial distance. The first secondary radial distance may be larger than 5 mm, such as larger than 6 mm, or larger than 8 mm. In one or more exemplary drug delivery devices, the first secondary radial distance is in the range from 6 mm to 15 mm.

In one or more exemplary drug delivery devices, the first attachment part, such as a part of the first needle and/or the first distal end, may, in the first state be arranged or at least partly arranged within a first primary recess of the first body part. In the first state, the first distal end may be arranged inside the first body part.

In one or more exemplary drug delivery devices, the first attachment part, such as a part of the first needle and/or the first distal end, may, in the second state be arranged or at least partly arranged outside the first primary recess of the first body part.

In one or more exemplary drug delivery devices, the first attachment part, such as a part of the first needle and/or the first distal end, may, in the first state be arranged within a second primary recess of the second body part. Thereby, the first attachment part may be configured to lock the first body part in relation to the second body part in the first state of the drug delivery device.

In one or more exemplary drug delivery devices, the first attachment part, such as a part of the first needle and/or the first distal end, may, in the second state be arranged outside the second body part and/or at least outside the second primary recess of the second body part.

In one or more exemplary drug delivery devices, the actuator mechanism is configured to move the second distal end from a second primary position, e.g. in first state of the drug delivery device, with a second primary radial distance from a central axis of the delivery device to a second secondary position, e.g. in second state of the drug delivery device, with a second secondary radial distance from the central axis and/or primary axis, wherein the second secondary radial distance is larger than the second primary radial distance. Thus, the second distal end of the second attachment may be in a second primary position when the drug delivery device is in the first state and/or the second distal end of the second attachment part may be in a second secondary position when the drug delivery device is in the second state.

The second primary radial distance may be less than 10 mm, such as less than 8 mm or even less than 5 mm. The second secondary radial distance may be larger than the second primary radial distance. The second secondary radial distance may be larger than 5 mm, such as larger than 6 mm, or larger than 8 mm. In one or more exemplary drug delivery devices, the second secondary radial distance is in the range from 6 mm to 15 mm.

In one or more exemplary drug delivery devices, the second attachment part, such as a part of the second needle and/or the second distal end, may, in the first state be arranged or at least partly arranged within a first secondary recess of the first body part. Thereby, the second attachment part may be configured to lock the first body part in relation to the second body part in the first state of the drug delivery device. In the first state, the second distal end may be arranged inside the second body part.

In one or more exemplary drug delivery devices, the second attachment part, such as a part of the second needle and/or the second distal end, may, in the second state be arranged or at least partly arranged outside the first body part and/or at least outside the first secondary recess of the first body part.

In one or more exemplary drug delivery devices, the second attachment part, such as a part of the second needle and/or the second distal end, may, in the first state be arranged within a second secondary recess of the second body part.

In one or more exemplary drug delivery devices, the second attachment part, such as a part of the second needle and/or the second distal end, may, in the second state be arranged outside the second secondary recess of the second body part.

In one or more exemplary drug delivery devices, the actuator mechanism is configured to move, e.g. by rotation about a first rotation axis of the first attachment part (first base part), the first distal end from a first primary angular position of first primary position to a first secondary angular position of first secondary position in relation to a first proximal end of the first attachment part. An angle between the first primary angular position and the first secondary angular position may be larger than 10°, such as larger than 45° or larger than 60°.

In one or more exemplary drug delivery devices, the actuator mechanism is configured to move, e.g. by rotation about a second rotation axis of the second attachment part (second base part), the second distal end from a second primary angular position of second primary position to a second secondary angular position of second secondary position in relation to a second proximal end of the second attachment part. An angle between the second primary angular position and the second secondary angular position may be larger than 10°, such as larger than 45° or larger than 60°.

In one or more exemplary drug delivery devices, the drug delivery device comprises a locking mechanism. The locking mechanism may be configured to lock, e.g. prevent rotation of, the first body part in relation to the second body part in a first state of the drug delivery device. The locking mechanism may be configured to lock the first attachment part in a first primary position, e.g. in relation to the first body part, when the drug delivery device is in the first state. Upon release of the locking mechanism, the first attachment part may be allowed to move from a first primary position to a first secondary position. The locking mechanism may upon release be configured to allow rotation of the first body part in relation to the second body part, e.g. in a second state of the drug delivery device. The locking mechanism may be configured to lock the second attachment part in a second primary position, e.g. in relation to the second body part, when the drug delivery device is in the first state. The locking mechanism may upon release be configured to allow the second attachment part to move from a second primary position to a second secondary position.

The locking mechanism may comprise a first locking element optionally configured to lock and/or unlock (release) the first body part in relation to the second body part. The first locking element may be configured to lock and/or unlock (release) the first attachment part in relation to the first body part. The first locking element may be configured to lock and/or unlock (release) the second attachment part in relation to the second body part. The first locking element may be arranged in a first primary recess of the first body part and/or in a second primary recess of the second body part. The first locking element may be configured to dissolve when the drug delivery device enters the gastrointestinal tract or at a desired location in the gastrointestinal tract, thereby releasing the first body part in relation to the second body part and allowing the actuator mechanism to rotate the first body part in relation to the second body part and thereby moving the first distal end towards the second distal end in turn resulting in attachment of the drug delivery device to the internal tissue.

The first locking element may be a first locking band (e.g., ring, loop, partial ring, partial loop). The first locking band may have a circumferential length greater than a longitudinal width. For example, the circumferential length may be 2×, 3×, 4×, 5×, 6×, 7×, 8×, 9×, or 10× the longitudinal width.

The first locking band may fit on an outer surface of the drug delivery device. For example, the first locking band may be located on an outer surface of the first body part or the second body part. The first locking band may be mechanically fit onto the drug delivery device. For example, the first locking band may be snap fit onto the drug delivery device. The first locking band may be chemically attached onto the drug delivery device.

In one or more exemplary drug delivery devices, the first locking band could be in the shape of a capsule part. For example, the first locking band could form a first half of a capsule. The first locking band could form a first half of a capsule and a second locking band could form a second half of the capsule. When fitted together, the first locking band and the second locking band could form a full capsule.

The first locking band may partially or fully cover the first body recess if located on the first body. Thus, the first locking band may prevent motion of the first attachment part. The first locking band may partially or fully cover the second body recess if located on the second body. Thus, the first locking band may prevent motion of the second attachment part. The first locking band may partially or fully cover both the first body recess and the second body recess. The first locking band may partially or fully cover the first body recess and a second locking band may partially or fully cover the second body recess.

The first locking band may extend fully along an outer circumference of the drug delivery device. The first locking band may extend around 10, 15, 20, 25, 30, 35, 40, 45, 50, 55, 60, 65, 70, 75, 80, 85, 90, 95, or 100% of an outer circumference of the drug delivery device. The first locking band may extend around greater than 10, 15, 20, 25, 30, 35, 40, 45, 50, 55, 60, 65, 70, 75, 80, 85, 90, or 95% of an outer circumference of the drug delivery device. The first locking band may extend around less than 10, 15, 20, 25, 30, 35, 40, 45, 50, 55, 60, 65, 70, 75, 80, 85, 90, 95, or 100% of an outer circumference of the drug delivery device.

In one or more exemplary drug delivery devices, at least part of the first attachment part and/or the second attachment part may be made of a biodegradable material, absorbable material, or similar material which allows the material of the attachment part to be broken down, degraded and/or dissolved by processes that are present in the body, such as corrosion, degradation, hydrolysis and/or proteolytic enzymatic degradation. Thus, when the attachment part(s) has been inside the human body for a period of time, the attachment part(s) may dissolve, decompose or degrade to such a degree that the attachment part may lose its structural stability, which may in turn release the drug delivery device from the surface it has attached itself to. Thus, after a period, e.g. when the drug substance has been released from the attachment part(s), the attachment part(s) may deteriorate to such a degree that the drug delivery device may be released and may continue its journey through the gastrointestinal tract to be released through natural intestinal and/or bowel movements of the user or patient.

In one or more exemplary drug delivery devices, the rotational axis of the first body part and/or the second body part (primary axis) may be the central axis of the drug delivery device, e.g. the primary axis of the first body part may be coaxial to the central axis. Thus, the central axis intersects both the first body part and the second body part, and may define the primary axis.

In one or more exemplary drug delivery devices, the first body part and the second body part may be substantially symmetrical in a radial direction perpendicular to the central axis. This may mean that the first body part and/or the second body part may have a circular periphery, where the periphery may extend in a radial direction away from and perpendicular to the central axis.

The first attachment axis may be seen as an axis that is coaxial with the length of the first attachment part. The second attachment axis may be seen as an axis that is coaxial with the length of the second attachment part. In case the first attachment part has a shape that is not straight, the first attachment axis may be defined as an axis that intersects the first distal end and the first proximal end of the first attachment part. In case the second attachment part has a shape that is not straight, the second attachment axis may be defined as an axis that intersects the second distal end and the second proximal end of the second attachment part.

In one or more exemplary drug delivery devices, the first attachment axis may be positioned at a first distance from the central axis, while the second attachment axis may be positioned at a second distance from the central axis and/or primary axis.

For example, the first attachment axis may be positioned at a first primary distance from the central axis in the first state of the drug delivery device. The first primary distance may be larger than 0.5 mm, such as in the range from 1 mm to 15 mm or larger than 1 mm, e.g. 2 mm, 3 mm, 4 mm, 5 mm, 6 mm, 7 mm, 8 mm, 9 mm, 10 mm, 11 mm, 12 mm, 13 mm, or 14 mm.

The first attachment axis may cross or be close to (distance less than 0.5 mm) the central axis in the first state of the drug delivery device.

The first attachment axis may be positioned at a first secondary distance from the central axis in the second state of the drug delivery device. The first secondary distance may be larger than 0.5 mm, such as in the range from 1 mm to 15 mm or larger than 1 mm, e.g. 2 mm, 3 mm, 4 mm, 5 mm, 6 mm, 7 mm, 8 mm, 9 mm, 10 mm, 11 mm, 12 mm, 13 mm, or 14 mm.

The first attachment axis may cross or be close to (distance less than 0.5 mm) the central axis in the second state of the drug delivery device.

For example, the second attachment axis may be positioned at a second primary distance from the central axis in the first state of the drug delivery device. The second primary distance may be larger than 0.5 mm, such as in the range from 1 mm to 15 mm or larger than 1 mm, e.g. 2 mm, 3 mm, 4 mm, 5 mm, 6 mm, 7 mm, 8 mm, 9 mm, 10 mm, 11 mm, 12 mm, 13 mm, or 14 mm.

The second attachment axis may cross or be close to (distance less than 0.5 mm) the central axis in the first state of the drug delivery device.

The second attachment axis may be positioned at a second secondary distance from the central axis in the second state of the drug delivery device. The second secondary distance may be larger than 0.5 mm, such as in the range from 1 mm to 15 mm or larger than 1 mm, e.g. 2 mm, 3 mm, 4 mm, 5 mm, 6 mm, 7 mm, 8 mm, 9 mm, 10 mm, 11 mm, 12 mm, 13 mm, or 14 mm.

The second attachment axis may cross or be close to (distance less than 0.5 mm) the central axis in the second state of the drug delivery device.

In one or more exemplary drug delivery devices, the first attachment part (first attachment axis) and/or the second attachment part (second attachment axis) may be configured to be at an angle to each other when they intersect a plane that includes the central axis. The plane may be a plane that contains the central axis, where the plane also includes a radial axis extending at a right angle from the central axis. When the first attachment part intersects the plane, the first attachment axis of the first attachment part may be at an angle to the plane so that the first distal end of the first attachment part is the first part of the first attachment part that intersects the plane, where the remaining parts of the first attachment part intersects the plane subsequently during rotational movement. The second attachment part may intersect the same plane from the opposite side, where the second distal end of the second attachment part is the first part of the second attachment part that intersects the plane, where the remaining parts of the second attachment part intersects the plane subsequently during rotational movement. Thus, the second attachment part optionally intersects the plane from an opposing rotational direction than the first attachment part. This may also mean that when the first distal end and the second distal end of respective first attachment part and second attachment parts each contact the plane, the first attachment part (first attachment axis) is at an angle to the plane as well as at an angle to the second attachment part (second attachment axis). The angle between the first attachment part (first attachment axis) and the second attachment part (second attachment axis) to the plane may be approximately half the size of the angle between the first attachment part and the second attachment part.

In one or more exemplary drug delivery devices, the first body part may be configured to rotate in a first direction and the second body part may be configured rotate in a second direction, where the first direction is opposite to the second direction. Thus, as an example, the first body part may rotate in a clockwise direction, while the second body part may rotate in an opposed anti clockwise direction. In one or more examples where the drug delivery device comprises three or more body parts, abutting or neighboring body parts may rotate in opposite directions. This may also mean that every second body part may rotate in the same direction. For example, where a first body part and a third body part rotate in the same first direction, then a second body part and/or a fourth body part may rotate in a second direction opposite the first direction.

In one or more exemplary drug delivery devices, the actuator mechanism may comprise one or more resilient parts, such as a plurality of resilient parts. The resilient parts can be configured to store energy. The resilient parts can be configured to convert the stored energy to kinetic energy.

In one or more exemplary drug delivery devices, the first distal end of the first attachment part and/or the second distal end of the second attachment part may be provided with a sharp tip configured to penetrate a biological tissue. The sharp tip may be positioned in the vicinity of the distal end of the respective attachment part, where the sharp tip may be configured to have a diameter at the distal end which is smaller than a diameter of the attachment part at a distance from the distal end. The sharp tip may be configured in such a manner that when a rotational force is applied to the first attachment part, and a counterforce is applied to the second attachment part, the counterforce may cause the sharp tip to penetrate the biological tissue due to the force applied by the actuator mechanism.

When the first attachment part and/or the second attachment part penetrates the biological tissue due to the rotation between the first body part and the second body part (the first distal end moving towards the second distal end, the respective penetration point(s) in the biological tissue may be utilized to deliver a drug substance from the drug delivery device into the biological tissue, and where the drug substance may be introduced into the biological tissue that is beyond the mucous membrane. Thereby, the drug substance may enter the bloodstream more easily than if the drug substance is released in the stomach or intestinal lumen, and the drug delivery may be more effective. An example of this is when the drug substance is insulin, where insulin may degrade inside the gastrointestinal tract and is not capable of being absorbed from the gastrointestinal tract, but where a mucous membrane has been penetrated, and the insulin released through the penetrated gastrointestinal wall, the insulin will remain intact and reach the bloodstream of the user via the blood vessels in the intestinal layer beyond the mucous membrane (surface).

In one or more exemplary drug delivery devices, the first attachment part and/or the second attachment part may be provided with a gripping part configured to grip a biological tissue. The gripping part may be utilized to improve traction between the attachment part and a mucous membrane, allowing the attachment part to anchor the drug delivery device inside the body of the user. The gripping part may be a part that increases a mechanical friction between the attachment part and the surface to be attached to, where the gripping part may e.g. have a hook shape, or e.g. a shape where the gripping part of the first attachment part faces the gripping part of the second attachment part, so that the biological tissue which is positioned between the first attachment part and the second attachment part is gripped between the two gripping parts.

In one or more exemplary drug delivery devices, a part of the resilient part may be connected to the first body part and a second part of the resilient part may be connected to the second body part. This means that the resilient part may be utilized to store energy, such as rotational energy, elastic potential energy, or rotational force which is applied to the first body part and the second body part, where the energy is stored in the resilient part. Furthermore, when the energy is released, e.g. when a locking element is dissolved or degraded, e.g. when the stored energy is converted into kinetic energy, the force may be released to both the first body part and the second body part, which in turn transfers the force to the first attachment part and the second attachment part. The resilient part may e.g. be in the form of a helical spiral spring (mainspring) and/or a spiral torsion spring, where the first body part may be wound relative to the second body part by rotating the first body part relative to the second body part. This stores energy in the mainspring by twisting the spiral tighter. The stored force of the mainspring may then rotate the first body part in the opposing direction as the mainspring unwinds. Thus, the force of the mainspring may cause the first attachment part and the second attachment parts to travel in opposing directions, and where the attachment parts may pinch the biological tissue and either pinch the tissue or penetrate the tissue in order to attach the drug delivery device to the biological tissue.

When the drug delivery device has entered the body, and has e.g. entered the desired part in the gastrointestinal tract, the drug delivery device may be configured to transform from the first state to the second state. The transformation may be initiated by different means, where e.g. the first and the second body parts may be held in the first state using a locking mechanism e.g. comprising one or more locking elements made of a dissolvable, expandable or degradable material, where the material reacts with the surroundings, such as fluids, inside the desired body part, thereby unlocking or releasing the locking mechanism. The material of the locking elements may be a material that loses its structural force when in contact with the surroundings inside the desired body part. An example may be where locking element(s) is made of a polymeric material or a sugary substance which may dissolve, expand or degrade when it comes into contact with a certain kind of fluid which may include an enzyme or a certain kind of acid inside the digestive system. When the locking element comes into contact with the reagent, the material may dissolve, expand or degrade over time, and when the rotational force of the drug delivery device exceeds the static force of the locking element, the rotational force may be released via a rotation of the first body part relative to the second body part, or vice versa.

In one or more exemplary drug delivery devices, a locking element may fix an attachment part in a position where the attachment element locks the first body part in relation to the second part, i.e. prevents the first body part from rotating in relation to the second body part. When the locking element dissolves or degrades, the attachment part can move to a secondary position where the attachment part does not lock the first body part in relation to the second part, e.g. by the actuator mechanism causing a rotation of the attachment part about a rotation axis in relation to the body part to which the attachment part is rotationally attached.

The second state of the drug delivery device may be seen as the state which is initiated by the release of energy stored, e.g. conversion of the stored energy into kinetic energy, in the actuator mechanism, e.g. resilient part(s) of the actuator mechanism into a rotational force of the first and/or the second body part and/or a rotational force of the first attachment part in relation to the first body part. A termination of the second state may be seen as a point in time where the energy stored in the resilient part becomes stationary again, i.e. when the attachment parts have gripped or penetrated biological tissue and/or the rotational movement between the first body part and the second body part is stopped.

In one or more exemplary drug delivery devices, the drug delivery device may have a first state where the actuator mechanism has a constant resilient force load and a second state where the actuator mechanism releases the resilient force load, e.g. converts the stored energy to kinetic energy. In the first state, the constant resilient force load may be seen as the energy stored in the actuator mechanism, and where the resilient force load is larger than zero. The second state may be seen as a state where the actuator mechanism releases its resilient force load, where the resilient force load is reduced, e.g. approaches zero, e.g. by rotating the first body part in relation to the second body part. The second state may be terminated when the attachment parts come into contact with or penetrates a biological tissue and the resilient force load does not change, even though it has not reached zero. Thus, a third state may follow the second state, when the drug delivery device has been attached to a wall of biological material, and the resilient force load is stationary after a resilient force release.

The first attachment part and/or the second attachment part may have an unfolding function, where during the first state of the drug delivery device, i.e. the initial state of the drug delivery device, the attachment parts are positioned or arranged inside the first and/or the second body part, or alternatively where the first and/or second attachment parts may be folded along the sides of the body parts. Other ways of obtaining the same may be envisioned. The folded state (first state) may e.g. be maintained using a releasable locking mechanism in the form of an encapsulation similar to a drug substance capsule, a band or plug, e.g. made of gelatine, sugars or other dissolvable materials or materials that lose their structural force. Thus, the attachment parts may be held in place until the drug delivery device has entered the gastrointestinal tract, e.g. the stomach, so that the attachment parts do not interfere or damage the lining of the mouth and/or the esophagus. Prior to or during the transition to the second state the attachment parts may extend from the body parts and outwards, making the attachment parts ready to interact with a lining of the digestive system. When the attachment part or parts are in a folded or collapsed position, the distance from the central axis to the distal end of the attachment part being longer in the second state than in a first state. Thus, the diameter of the drug delivery device in the first state will be less in than the diameter of the drug delivery device in the second state.

In one or more exemplary drug delivery devices, at least part of the first attachment part and/or the second attachment part, such as the first needle and/or the second needle may be made of material comprising one or more of magnesium, titanium, iron and zinc which allows for accurate and precise control of the size and/or shape/geometry of the first attachment part and/or second attachment part in turn allowing for a delivery device with desired attachment capabilities and/or small production variances which is in particular important in the pharmaceutical industry.

The first attachment part, such as the first needle, may be made of material comprising one or more of magnesium, titanium, iron and zinc. The material of the first attachment part/first needle may be biocompatible and/or biodegradable such as a biocompatible material and/or a biodegradable material. The material of the first attachment part/first needle may comprise one or more biodegradable polymers such as PLA and/or POLGA. Some of, part of, most of, substantially all, or all of the material of the first attachment part/first needle may be biocompatible and/or biodegradable. The material of the first attachment part, such as the first needle, may comprise, consist of, or essentially consist of, biocompatible and/or biodegradable material such as biocompatible and/or biodegradable metals. The material of the first attachment part, such as the first needle, may comprise a biodegradable or bioresorbable metal or metal alloy, such as magnesium, zinc, and/or iron or an alloy comprising one or more of magnesium, zinc and iron. A biodegradable or bioresorbable metal or metal alloy may be understood as a metal or metal alloy that degrades safely within e.g. a human body in a practical amount of time, for example related to their application. The material of the first attachment part, such as the first needle, may comprise one or more metals such as a combination of one or more metals e.g. as a metal alloy.

The second attachment part, such as the second needle, may be made of material comprising one or more of magnesium, titanium, iron and zinc. The material of the second attachment part/second needle may be biocompatible and/or biodegradable such as a biocompatible material and/or a biodegradable material. The material of the second attachment part/second needle may comprise one or more biodegradable polymers such as PLA and/or POLGA. Some of, part of, most of, substantially all, or all of the material of the second attachment part/second needle may be biocompatible and/or biodegradable. The material of the second attachment part, such as the second needle, may comprise, consist of, or essentially consist of, biocompatible and/or biodegradable material such as biocompatible and/or biodegradable metals. The material of the second attachment part, such as the second needle, may comprise a biodegradable or bioresorbable metal or metal alloy, such as magnesium, zinc, and/or iron or an alloy comprising one or more of magnesium, zinc and iron. A biodegradable or bioresorbable metal or metal alloy may be understood as a metal or metal alloy that degrades safely within e.g. a human body in a practical amount of time, for example related to their application. The material of the second attachment part, such as the second needle, may comprise one or more metals such as a combination of one or more metals e.g. as a metal alloy.

An advantage of having a biodegradable material used in the attachment part(s) may be that the delivery device is able to deliver an active drug substance or payload arranged in the attachment part(s) and/or body parts of the delivery device at a specific part of the body of the subject, e.g. such as the stomach or intestines after the delivery device has attached to the internal surface, e.g. the intestinal wall, thanks to the sharp properties of the material of the attachment part(s), and for an extended period of time, since the biodegradable material will degrade gradually in time. Further, when the material of the attachment part(s) is biodegradable, the attachment part(s) will degrade in the human body and disappear after having delivered the payload/active drug substance comprised in the drug delivery device, thereby avoiding harming the human subject over time. The attachment part(s) may be configured to degrade in a period of time of hours, e.g. 2 hours, 5 hours, 10 hours, 20 hours, or 24 hours, days, e.g. 1 day, 2 days, 5 days, or weeks, e.g. 1 week, 2 weeks, 3 weeks, or 5 weeks.

The material of the attachment part(s), such as the needle(s), may comprise one or more or a combination of magnesium (Mg), zinc (Zn), and/or iron (Fe). An advantage of having the attachment part(s) of a material comprising Mg, Zn, and/or Fe may be that the shape and size of the attachment part(s) can be precisely controlled thereby providing improved attachment to the internal surface, for example to an internal wall of the intestines of the human subject.

The attachment part(s), such as the needle(s), may be made of a material comprising one or more thermoplastic or thermoset polymers. The material of the attachment part(s), such as the needle(s), may comprise one or more active drug substances. Thus, an active drug substance may be embedded in the material of the attachment part(s), such as the needle(s), to form a pharmaceutical composition.

In some embodiments, the attachment part(s), such as the needle(s), may comprise for example water soluble, water insoluble, biodegradable, non-biodegradable and/or pH dependent soluble materials. In some embodiments, the attachment part(s), such as the needle(s), may comprise a water soluble, biodegradable and/or pH-dependent material that may dissolve and/or degrade so that the attachment part(s), such as the needle(s), lodged in the intestinal tissue may gradually degrade and/or dissolve. In some embodiments, the attachment part(s), such as the needle(s), may comprise a water-soluble material to allow immediate release or modified release of the active drug substance depending of the material selected. In some embodiments, a water insoluble or biodegradable material may allow depot of the active drug substance in the attachment part(s), such as the needle(s), for longer release duration (for example days, weeks or months). In some embodiments, a pH dependent soluble material may allow the attachment part(s), such as the needle(s), to stay intact at pH conditions below the physiologic for example a pH of approximately 7.4 to remain intact in the gastrointestinal lumen, but then may dissolve once inside the gastrointestinal wall. In some embodiments, one or more water soluble, water insoluble, biodegradable and/or pH dependent materials may optionally be combined to control release of the active drug substance for example by diffusion or erosion of the attachment part(s), such as the needle(s), for controlled release duration (for example minutes, hours, days, weeks, or months).

In some embodiments, the attachment part(s), such as the needle(s), may be made from different compositions. For example, an outer part of the attachment part(s), such as the needle(s), may be made of one composition and an inner core of the attachment part(s), such as the needle(s), may be made from another composition. In some embodiments, the outer part and the inner core of the attachment part(s), such as the needle(s), may be composed of for example a water soluble, a water insoluble, a biodegradable, and/or a pH dependent material. In some embodiments, one or more water soluble, water insoluble, biodegradable and/or pH dependent materials may be combined to control the release of the active drug substance once the attachment part(s), such as the needle(s), may move its position from the lumen to the internal tissue for example the gastrointestinal lumen to the gastrointestinal tissue.

In some embodiments, the attachment part(s), such as the needle(s), may be tubular and may include a tubular body and the tubular body may comprise an active drug substance for example a liquid payload comprising the active drug substance, optionally connected to a tubular attachment part so the payload with the active drug substance may flow though the attachment part(s), such as the needle(s), into the internal tissue for example the intestinal tissue. In some embodiments, the tubular body may contain expandable such as swelling excipients that may expand by a chemical reaction for example when mixed expand in volume and/or produce a gas to advance the delivery of the payload. In some embodiments, the expansion is by osmosis.

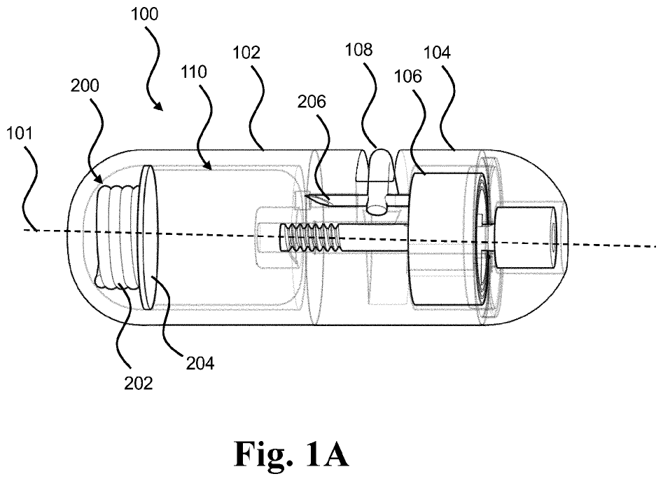

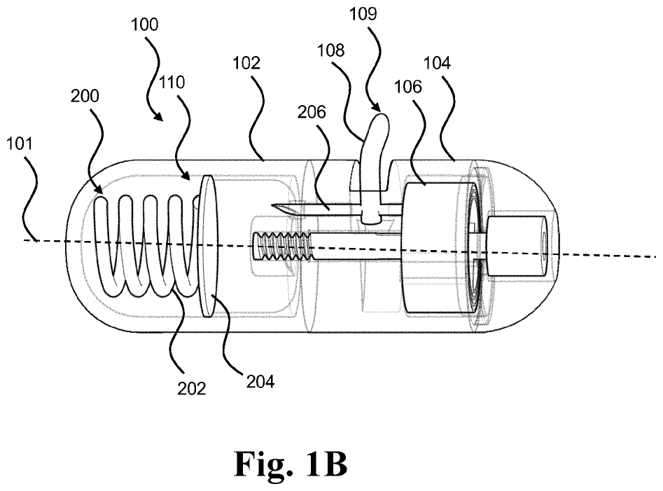

FIG. 1A-1B illustrate an example high-dose drug delivery device according to the disclosure. As shown, the high-dose drug delivery device 100 can include a first body part 102 and a second body part. The high-dose drug delivery device can further include an actuator mechanism 106. The actuator mechanism 106 can be configured to rotate at least one of the first body part 102 or the second body part 104 with respect to one another about a central axis 101 of the high-dose drug delivery device 100.

The high-dose drug delivery device 100 can further include an internal chamber 110. As shown, the internal chamber can be located within the first body part 102. Alternatively, the internal chamber 100 can located in the second body part 104, or in both the first body part 102 and the second body part 104. The internal chamber 110 can have a volume of at least 5 μL. The internal chamber 110 can have a volume of at least 1 mL. The internal chamber 110 can be configured to releasably retain an active drug substance. The internal chamber 110 can be configured to releasably retain 1 μg-500 mg of the active drug substance.

The high-dose drug delivery device 100 can further include a first attachment part 108 in communication with the internal chamber 110. For example, the communication can be a fluid communication. The first attachment part 108 can include a lumen extending between the internal chamber 110 and an outside of the high-dose drug delivery device 100. The first attachment part 108 can include a channel 109 fluidly connecting the internal chamber 110 and an outer surface of the first attachment part 108.

The high-dose drug delivery device 100 can further include an injector mechanism 200. The injector mechanism 200 can be configured to apply pressure, such as a compressive force, on the internal chamber 110, or portions of the internal chamber 110, during or after rotation of the first body part 102 or the second body part 104 with respect to one another. The internal chamber 110 may be under pressure prior to any rotation. The internal chamber 110 may be under pressure prior to use of the injection mechanism 200.

FIGS. 1A-1B illustrate an injector mechanism 200 comprising a compression spring. As shown, the injector mechanism can be a compression spring 202, which may be, for example, optionally be attached to a platform 202. The compression spring 202 can be attached to an internal surface of the internal chamber 110, or another component of the high-dose drug delivery device 100.

As shown in FIG. 1A, the compression spring 202 can start in a compressed state within the internal chamber 110. Thus, the platform 202 can be spaced far away from a longitudinal center of the high-dose drug delivery device. This allows the internal chamber 110 to have a full volume.

Due to the actions of the actuator mechanism 106, the compression spring 202 can be allowed to un-compress. This can move the platform 202 towards the longitudinal center of the high-dose drug delivery device 100.

In certain implementations, the uncompressing of the compression spring 202, such as shown in FIG. 1B, can reduce the overall volume of the internal chamber 110, thereby increasing pressure within the internal chamber 100 and forcing the active drug substance out of the internal chamber.

Further, uncompressing the compression spring 202 can translate a container containing an active drug substance. For example, the container can be attached to the platform 204 or the compression spring 202. The internal chamber 110 may optionally include a spike 204, such as a needle, which can pierce and/or break the container as the compression spring 206 translates the container into the spike 206. Thus, the injector mechanism 200 can include a spike 206 configured to pierce a container.

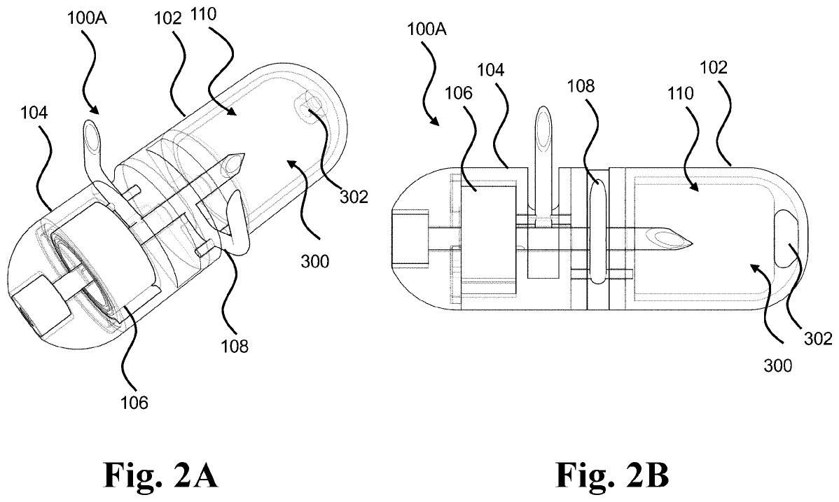

FIGS. 2A-2B illustrate an example high-dose drug delivery device according to the disclosure. The high-dose drug delivery device 100A of FIGS. 2A-2B can include any and/or all components discussed above with respect to high-dose drug delivery device 100.

FIGS. 2A-B illustrate an injector mechanism 300 comprising an expandable gas. As shown, the expandable gas can be stored within an expandable container 302. As the expandable container 302 expands, due to the expansion of the gas, it reduces the volume of the internal chamber 100, thereby increasing pressure within the internal chamber 100. This can force an active drug substance out of the internal chamber 110.

Optionally, the injector mechanism 300 can include a platform 304 attached to the expandable container 302. The platform 304 can be translated due to the expansion of the gas, and thus the expandable container 302, to reduce the volume of the internal chamber.

Further, expanding the expandable container 302 can translate a container containing an active drug substance. The internal chamber 110 may optionally include a spike, such as a needle, which can pierce and/or break the container as expandable container 302 expands.

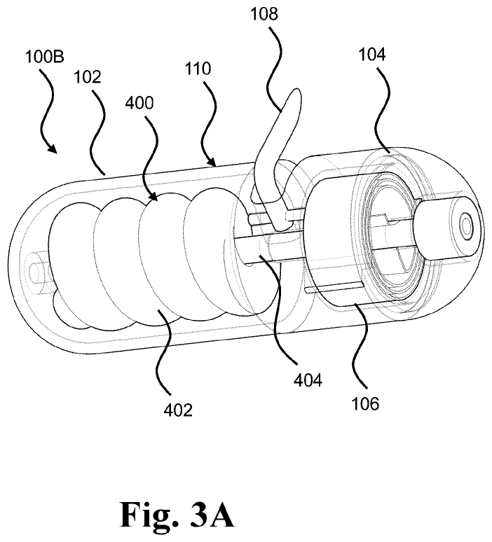

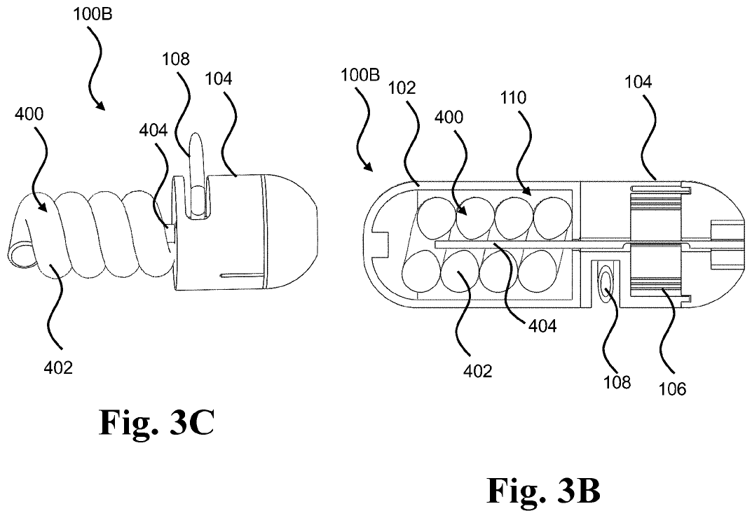

FIGS. 3A-3C illustrate an example of a high-dose drug delivery device according to the disclosure. The high-dose drug delivery device 100B of FIGS. 3A-3C can include any and/or all components discussed above with respect to high-dose drug delivery device 100 and 100A.

FIGS. 3A-3C illustrate an injector mechanism 400 comprising a swellable polymer. As shown, the swellable polymer 402 can be located within the internal chamber 110. FIGS. 3A-3C illustrate the swellable polymer 402 wrapped around a rod 404, though other configurations can be used as well.

As the swellable polymer 402 swells, it reduces the volume of the internal chamber 100, thereby increasing pressure within the internal chamber 100. This can force an active drug substance out of the internal chamber 110.

FIG. 3C illustrates the first body part 102 removed for clarity.

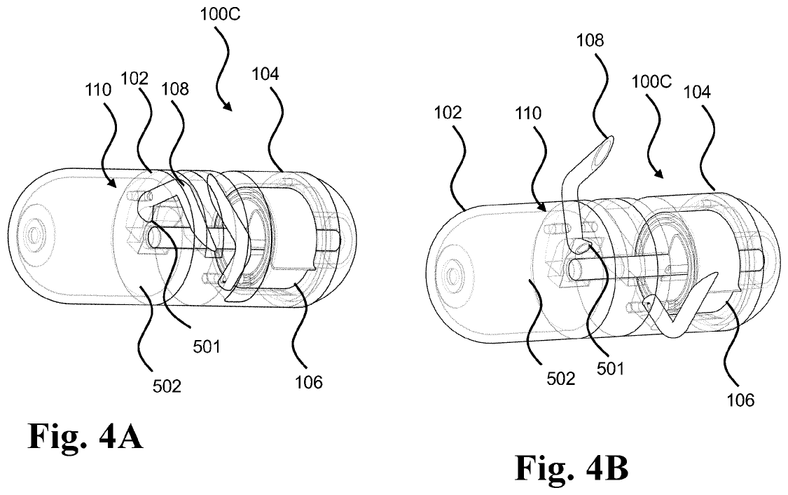

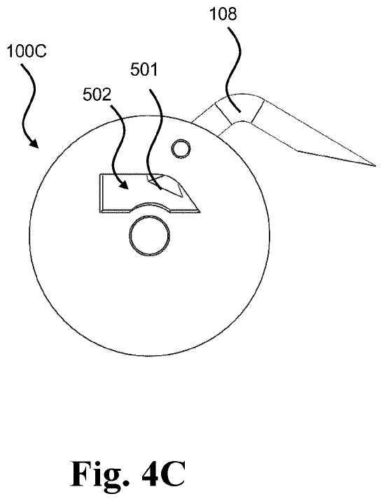

FIGS. 4A-4C illustrate an example of a high-dose drug delivery device according to the disclosure. The high-dose drug delivery device 1000 of FIGS. 4A-4C can include any and/or all components discussed above with respect to high-dose drug delivery device 100, 100A, and 100B.

As shown, the attachment part 108 can include an edge 501, such as a cutting, slicing, and/or piercing edge. As the attachment part 108 rotates, the edge 109 can penetrate a container 502, such as a pouch or bag, within the internal chamber 110. This can release an active drug substance from the container 502. The container 502 may be pressurized, thus quickly releasing the active drug substance.

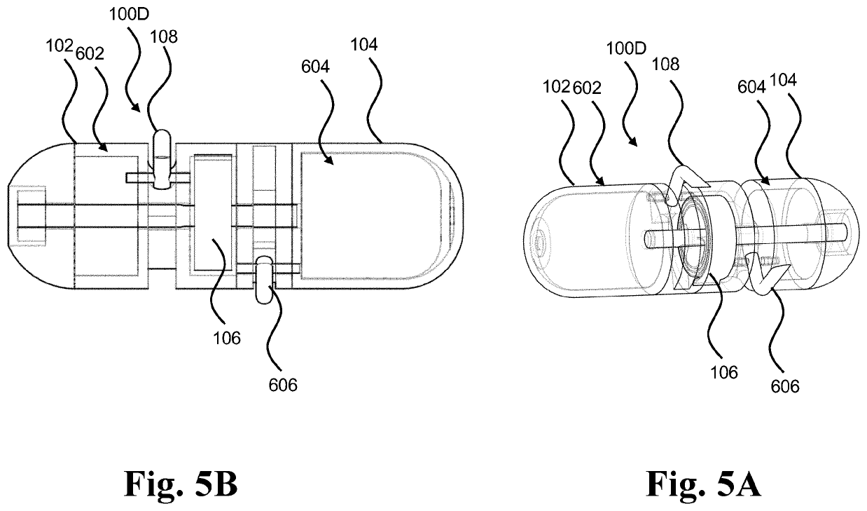

FIGS. 5A-5B illustrate an example of a high-dose drug delivery device according to the disclosure. The high-dose drug delivery device 100D of FIGS. 5A-5B can include any and/or all components discussed above with respect to high-dose drug delivery device 100, 100A, 100B, and 100C.

The high-dose drug delivery device 100D can include any of the injector mechanisms discussed above. FIGS. 5A-5B illustrate a high-dose drug delivery device 100D which can include two internal chambers and two attachment parts.

In particular, the drug delivery device 100D can include a first a first internal chamber 602 in the first body part 102. The first internal chamber 602 can be in fluid communication with the first attachment part 108, such as after rotation. The drug delivery device 100D can include a second internal chamber 604 in the second body part 104. The second internal chamber 604 can be in fluid communication with the second attachment part 606, such as after rotation.

Advantageously, this allows for two different active drug substances to be stored in the drug delivery device 100, one in the first internal chamber 602 and one in the second internal chamber 604. Further, each chamber can have a different injection mechanism, which can allow for different dosing strategies. Further, each chamber can have the same active drug substance, but may deliver at different times due to different injection mechanisms.

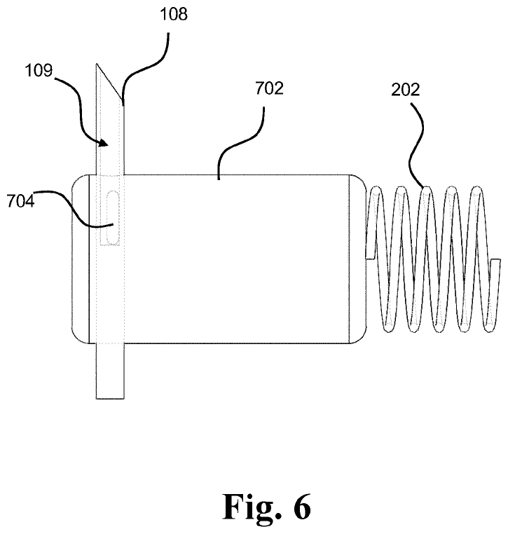

FIG. 6 illustrates an example of a solid active drug substance in a high-dose drug delivery device. For example, the high-dose drug delivery device can be any of the high-dose drug delivery devices 100, 100A, 100B, 100C, or 100D disclosed above, and can include any and/or all of the components discussed with respect to them. FIG. 6 illustrates a compression spring 202, and therefore will be discussed with respect to high-dose drug delivery device 100. The drug delivery device 100 can further include a container 702, which can hold an active drug substance. The container 702 may be within the internal chamber 110 discussed herein.

The compression spring 202 can be activated in order to increase the pressure in the chamber 702. When the attachment part 108 passes through the container 702, such as pierces the container 702, the active drug substance can enter a channel 109 in the attachment part 108 through aperture 704.

Alternatively, the attachment part 108 may not pierce the container 702, but may instead pass through the internal chamber 110. When the aperture 704 is aligned with the internal chamber 110, providing fluid communication, the active drug substance can exit the internal chamber 110 and pass through the attachment part 108.

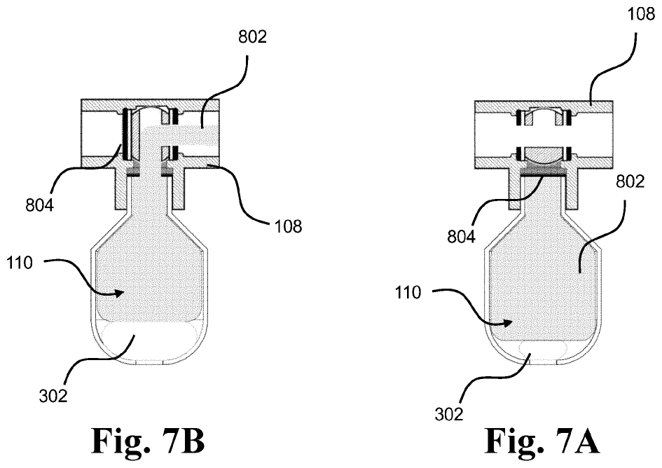

FIGS. 7A-7B illustrate an example release mechanism for use in a high-dose drug delivery device. For example, the high-dose drug delivery device can be any of the high-dose drug delivery devices 100, 100A, 100B, 100C, or 100D disclosed above, and can include any and/or all of the components discussed with respect to them. FIGS. 7A-7B illustrate a expandable container 302, and therefore will be discussed with respect to high-dose drug delivery device 100A.

As shown, the active drug substance 802 can be stored within an internal chamber 110. The active drug substance 802 could be a liquid and/or suspension. The expandable container 302 can be at its lowest expansion as shown in FIG. 7A. The active drug substance 802 is prevented from releasing into the first attachment part 108 via a valve 804. The valve 804 can be a ballo-fix valve.

As the expandable container 302 expands, it can pressurize the internal chamber 110. This can, for example, open the valve 804. Alternatively, rotation of the first body part 102 with respect to the second body part 104 can open the valve 804 to provide a fluid pathway into the first attachment part 108, such as through channel 109. As the internal chamber 110 is under pressure, this can quickly inject the active drug substance.

Also disclosed are high-dose drug delivery devices, methods, and compositions according to any of the following items.

Item 1. A high-dose drug delivery device comprising:
a first body part;
a second body part;
an internal chamber within the first body part;
an actuator mechanism configured to rotate at least one of the first body part or the second body part with respect to one another about a central axis of the high-dose drug delivery device;
a first attachment part in communication with the internal chamber; and
an injector mechanism configured to apply pressure on the internal chamber during or after rotation of the first body part or the second body part with respect to one another.

Item 2. High-dose drug delivery device according to Item 1, wherein the injector mechanism comprises a swellable polymer.

Item 3. High-dose drug delivery device according to any one of the preceding Items, wherein the injector mechanism comprises an expandable gas.

Item 4. High-dose drug delivery device according to any one of the preceding Items, wherein the injector mechanism comprises a breakable seal.

Item 5. High-dose drug delivery device according to any one of the preceding Items, wherein the injector mechanism comprises a first component separated from a second component, and wherein the rotation interacts the first component with the second component to form an expandable gas.

Item 6. High-dose drug delivery device according to Item 5, wherein the first component is separated from the second component via a pH releasable plug.

Item 7. High-dose drug delivery device according to any one of the preceding Items, wherein the injector mechanism comprises a spike configured to pierce a container.

Item 8. High-dose drug delivery device according to any one of the preceding Items, wherein the injector mechanism comprises a compression spring.

Item 9. High-dose drug delivery device according to any one of the preceding Items, wherein the internal chamber is under pressure prior to the rotation.

Item 10. High-dose drug delivery device according to any one of the preceding Items, wherein the first attachment part comprises a channel fluidly connecting the internal chamber and an outer surface of the first attachment part.

Item 11. High-dose drug delivery device according to any one of the preceding Items, wherein the internal chamber has a volume of at least 5 μL.

Item 12. High-dose drug delivery device according to any one of the preceding Items, wherein the internal chamber has a volume of at least 1 mL.

Item 13. High-dose drug delivery device according to any one of the preceding Items, wherein the internal chamber is configured to releasably retain an active drug substance.

Item 14. High-dose drug delivery device according to Item 13, wherein the active drug substance is a solid rod.

Item 15. High-dose drug delivery device according to Item 13 or Item 14, wherein the active drug substance is a liquid and/or a suspension.

Item 16. High-dose drug delivery device according to any one of Items 13-15, wherein the internal chamber is configured to releasably retain 1 μg-500 mg of the active drug substance.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Furthermore, the labelling of a first element does not imply the presence of a second element and vice versa.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the exemplary embodiments may be implemented at least in part by means of both hardware and software, and that several “means”, “units” or “devices” may be represented by the same item of hardware.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

### LIST OF REFERENCES

100 high-dose drug delivery device
101 central axis
102 first body part
104 second body part
106 actuator mechanism
108 first attachment part
109 channel
110 internal chamber
200 injector mechanism
202 compression spring
204 platform
206 spike
100A high-dose drug delivery device
300 injector mechanism
302 expandable container
304 platform
100B high-dose drug delivery device
400 injector mechanism
402 swellable polymer
404 rod
1000 high-dose drug delivery device
501 edge
502 container
100D high-dose drug delivery device
602 first internal chamber
604 second internal chamber
606 second attachment part
702 container
704 aperture
802 active drug substance
804 valve

1. A high-dose drug delivery device comprising:
a first body part;
a second body part;
an internal chamber within the first body part;
an actuator mechanism configured to rotate at least one of the first body part or the second body part with respect to one another about a central axis of the high-dose drug delivery device;
a first attachment part in communication with the internal chamber; and
an injector mechanism configured to apply pressure on the internal chamber during or after rotation of the first body part or the second body part with respect to one another.

2-16. (canceled)

17. The high-dose drug delivery device according to claim 1, wherein the injector mechanism comprises a swellable polymer.

18. The high-dose drug delivery device according to claim 1, wherein the injector mechanism comprises an expandable gas.

19. The high-dose drug delivery device according to claim 1, wherein the injector mechanism comprises a breakable seal.

20. The high-dose drug delivery device according to claim 1, wherein the injector mechanism comprises a first component separated from a second component, and wherein the rotation interacts the first component with the second component to form an expandable gas.

21. The high-dose drug delivery device according to claim 20, wherein the first component is separated from the second component via a pH releasable plug.

22. The high-dose drug delivery device according to claim 1, wherein the injector mechanism comprises a spike configured to pierce a container.

23. The high-dose drug delivery device according to claim 1, wherein the injector mechanism comprises a compression spring.

24. The high-dose drug delivery device according to claim 1, wherein the internal chamber is under pressure prior to the rotation.

25. The high-dose drug delivery device according to claim 1, wherein the first attachment part comprises a channel fluidly connecting the internal chamber and an outer surface of the first attachment part.

26. The high-dose drug delivery device according to claim 1, wherein the internal chamber has a volume of at least 5 μL.

27. The high-dose drug delivery device according to claim 1, wherein the internal chamber has a volume of at least 1 mL.

28. The high-dose drug delivery device according to claim 1, wherein the internal chamber is configured to releasably retain an active drug substance.

29. The high-dose drug delivery device according to claim 28, wherein the active drug substance is a solid rod.

30. The high-dose drug delivery device according to claim 28, wherein the active drug substance is a liquid and/or a suspension.

31. The high-dose drug delivery device according to claim 28, wherein the internal chamber is configured to releasably retain 1 μg-500 mg of the active drug substance.
